(19) United States
(12) Patent Application Publication
(10) Pub. No.: US 2022/0265646 A1
(43) Pub. Date: Aug. 25, 2022

(54) PIEZO AGONISTS FOR PREVENTING OR REVERTING ABNORMAL AMYLOID DEPOSITION

(71) Applicant: ITÄ-SUOMEN YLIOPISTO, Kuopio (FI)

(72) Inventors: Tarja MALM, Siilinjärvi (FI); Rashid GINIATULLIN, Kuopio (FI)

(73) Assignee: ITÄ-SUOMEN YLIOPISTO, Kuopio (FI)

(21) Appl. No.: 17/625,004

(22) PCT Filed: Jul. 7, 2020
(86) PCT No.: PCT/FI2020/050483

§ 371 Date: Jan. 5, 2022

(30) Foreign Application Priority Data
Jul. 8, 2019 (FI) 20195618

(51) Int. Cl. A61K 31/497; A61P 25/28; A61K 31/341; A61K 31/381; A61K 31/164; A61K 31/232; A61P 29/00; A61P 9/10

(52) U.S. Cl. CPC A61K 31/497; A61P 25/28; A61K 31/341; A61K 31/381; A61K 31/164; A61K 31/232; A61P 29/00; A61P 9/10

(57) ABSTRACT

The present invention relates to diagnosing, preventing, delaying or reverting the progression of pathologies associated with abnormal amyloid deposits, such as that exemplified by Alzheimer's disease (AD). More specifically, the method involves administration of specific molecules that function as Piezo agonists, such as Yoda1, Jedi1, 5 Jedi2, or functional analogs thereof, that are able to modulate microglial activation towards anti-inflammatory state and/or interfere with the formation of amyloidogenic peptides and/or increase their efflux from the central nervous system. These agonists are applicable in disease states associated with, or at risk of, cerebral amyloidosis, such as AD, Parkinson's disease, stroke, head trauma(s), cerebral amyloid angiopathies, spongiform 10 encephalopathies and scrapie all of which are evidenced with abnormal proinflammatory microglial activation

### FIELD OF THE DISCLOSURE

The present invention relates to diagnosing, preventing, delaying or reverting the progression of pathologies associated with abnormal amyloid deposits, such as that exemplified by Alzheimer's disease (AD). More specifically, the method involves administration of specific molecules that function as Piezo agonists, such as Yoda1, Jedi1, Jedi2, or functional analogs thereof, that are able to modulate microglial activation towards anti-inflammatory state and/or interfere with the formation of amyloidogenic peptides and/or increase their efflux from the central nervous system. These agonists are applicable in disease states associated with, or at risk of, cerebral amyloidosis, such as AD, Parkinson's disease, head trauma(s), stroke, cerebral amyloid angiopathies, spongiform encephalopathies and scrapie all of which are evidenced with abnormal proinflammatory microglial activation.

### BACKGROUND OF THE DISCLOSURE

Microglia are highly dynamic cells that chemically and mechanically interact with their environment. Sensitive to their environment changes in human brain they balance between pro- and anti-inflammatory phenotype (Hammond, Robinton, and Stevens 2018; Malm, Jay, and Landreth 2015). Reshaping microglia towards the beneficial phenotype represents one of promising strategies for improving brain functions in the neurodegenerative diseases such as Alzheimer's disease (AD). Where one of the classic hallmarks is accumulation of microglia around beta-amyloid or amyloid-β (Aβ) plaques (Maim et al. 2015). The functional state of these immune cells surrounding plaques, particularly their motility, can be a key determinant of the pathological process in AD. While the impact of chemical signalling on microglia function has been broadly studied, the current knowledge of mechanical signalling is very limited. Especially due to ability of neurodegenerative disorders to change the mechanical properties of the brain, such as stiffness, which is known to decrease in human brain with the AD development (ElSheikh et al. 2017; Murphy et al. 2016). Amyloid-β plaques formation is increasing as a local stiffness due to its fibrillar nature (˜3×109 Pa) compare to normal brain tissues stiffness (˜200-500 Pa), what introduces stiffens gradient in the brain (Moeendarbary et al. 2017; Smith et al. 2006). As active as microglia is, this cell type strongly reacts to mechanical changes in their surrounding and have been described to change their morphology on stiffer substrates and increase mobility towards increase of the stiffens gradient (Bollmann et al. 2015; Moshayedi et al. 2014). Similarly, to astrocytes, microglia upregulate inflammatory mediators when attracted to the region with increased stiffness and increases their inflammatory responses (Bollmann et al. 2015; Moshayedi et al. 2014). This important function of microglia along heterogeneous extracellular environment, associated with cell reshaping, should be dependent on or ultimately lead to activation of mechanosensitive channels. These newly discovered mechanosensitive Piezo receptors fast-gained interest and have already been demonstrated to shape astroglial responses to mechanical stimuli of extracellular Aβ plaques (Velasco-Estevez et al. 2018). However, neither expression nor a functional role of mechanosensitive channels in brain residing microglia have been explored so far.

Mechanosensitive (MS) ion channels are molecular force transducers that are specialized to rapidly convert various mechanical forces into electrochemical signals for controlling key biological activities such as touch perception, hearing and blood pressure regulation. It is thus imperative to understand how this conversion process, termed mechanogating, precisely occurs. While significant progresses have been made in studying prokaryotic MS channels (i.e. MscL), relatively little is known about the mechanogating mechanisms of mammalian MS cation channels.

Mechanosensitive Piezo receptors have emerged recently as the most specific transducers of mechanical stimuli ever discovered (Coste et al. 2010; Coste et al. 2012). Mechanically sensitive cell types were found not only in specialized organs such as auditory or vestibular system, but also in other deformable excitable and non-excitable tissues throughout the body (Coste et al. 2010). The evolutionarily conserved Piezo family of proteins, including Piezo1 and Piezo2, has been established as the long-sought mammalian MS cation channels. Piezo2 receptor (Piezo2Rs) subtype is mainly expressed in the nociceptive system (Bron et al. 2014; Eijkelkamp et al. 2013; Kim et al. 2012), while Piezo1 channels are present both at periphery and in various brain regions (Velasco-Estevez et al. 2018; Wu et al. 2017). Simultaneously with this study, Philip A. Gottlieb laboratory found that soluble Aβ blocks Piezo1 receptor (Piezo1 Rs) (Maneshi et al. 2018), suggesting a close link between AD pathology and functional state of brain cells expressing these mechanosensitive channels.

In mice, Piezos have been shown to play critical roles in various mechanotransduction processes, including the sensation of touch, hearing and blood flow-associated shear stress. In humans, mutations of Piezo genes resulting in altered channel functions have been linked to a number of genetic diseases involving mechanotransduction. These studies demonstrate the functional importance and the potential as therapeutic targets of Piezo channels. The Piezo channel represents a prototype of mammalian mechanosensitive cation channels. However, its mechanogating mechanisms remain unclear.

The chemical agonist Yoda1 specifically activates (and modulates) Piezo1 channels in Ca²⁺-imaging assays, providing a simple method for uniform stimulation of a large population of channels. The precise mechanism by which Yoda1 activates Piezo1 remains unknown; the open state stabilized by Yoda1 has an identical single-channel conductance to the tension-gated open state, suggesting both pore open conformations are similar. There are several reports suggesting the beneficial effects of Yoda1 in various body functions such as vasodilation (Li et al. 2014; Wang et al. 2016), activation of the neuroprotective Ark pathways (dela Paz and Frangos 2018). In addition, Yoda1 induced stimulation of the endothelial release of ATP (Wang et al. 2016) can contribute to the indirect activation of these brain resident immune cells highly sensitive to released extracellular ATP.

WO2018232735A1 discloses novel Piezo1 chemical activators, termed Jedi that directly bind to and activate Piezo1 via modulating its mechanosensitivity. EP3006055 discloses a neural implant comprising a biomaterial having an outer surface with a stochastic nanoroughness, and the application of said stochastic nanoroughness in the diagnosis and/or treatment of a neurological disorder and for disrupting or preventing glial scars in the context of mammalian mechanosensing ion channels. Blumenthal et al. 2014 teaches that inhibition of mechanosensing cation channels including Piezo-1, whose distribution is altered by nanotopography, abrogates the effects imposed by nanotopography and the association of neurons with astrocytes.

AD, the most common form of dementia in late life, will become even more prevalent by midcentury, constituting a major global health concern with huge implications for individuals and society. Despite scientific breakthroughs during the past decades that have expanded our knowledge on the cellular and molecular bases of AD, therapies that effectively halt disease progression are still lacking, and focused efforts are needed to address this public health challenge. Stroke, the third leading cause of death and disabilities in industrialised countries, similarly lacks effective treatment strategies. These diseases cause a major socioeconomical burden not only to those affected, but also to their relatives and caretakers. Pathologically brain diseases, such as AD, stroke, Parkinson's disease as examples are all manifested by abnormal microglial activation and abnormal protein accumulation either in the extracellular space or intracellularly as a primary (AD, Parkinson's disease) or secondary (stroke, head trauma) pathology.

### BRIEF DESCRIPTION OF THE DISCLOSURE

This is the first time that Piezo receptors expression in microglia and their emerging role in the regulation of its important functions as motility, phagocytosis and cytokine release is demonstrated. So far evidence of the microglial role as one of the central players in the AD origination and progression is a point of a debate. On one hand, microglia are involved in AD pathogenesis by implicating to the inflammation by release of the inflammatory mediators (cytokines, chemokines, free radicals) that are known to contribute to beta-amyloid (Aβ) accumulation and promote nearby neuronal death (Cai, Hussain, and Yan 2014; Parkhurst et al. 2013). On the other hand, microglia are also known to play a beneficial role in generating stimulating clearance of amyloid plaques and anti-Aβ antibodies (Perlmutter et al. 1992). Thus, reappearing as a problem of ‘chicken or the egg’, Aβ plagues accumulation and clearing in which AD microglia both participates.

The present inventors hypothesized, that the presence of Piezo1Rs channels in microglia which play a central role in AD associated neuroinflammation could be targeted to clean brain from Aβ plaques. The discovered failure of microglia to clean the brain in the early onset of the AD is due to its functional inhibition by the Aβ via Piezo1Rs, which is in alignment with Maneshi et al. 2018. The present results suggest that both microglia from mice and human samples express Piezo1Rs channels and their activation by Yoda1 largely reduces the size of Aβ plagues.

In sum, the present invention identifies a specific molecule that is unexpectedly able to interfere with the formation of amyloidogenic Aβ peptides and/or increase their efflux from the central nervous system and, as such, able to reduce the load of Aβ deposition in brain in disease states associated with, or at risk of, cerebral amyloidosis, such as AD, cerebral amyloid angiopathies, spongiform encephalopathies and scrapie. Moreover, the present invention identifies this molecule to modulate microglial activation status to less proinflammatory and more phagocytic and thereby provide protection in diseases in which aberrant microglial activation persists. Moreover, the present invention refers to the use of the above-mentioned compound. The present invention also relates to uses of compositions of the molecule in association with other pharmacologically active substances capable of affecting amyloid burden in brain, such as antioxidants, anti-inflammatory agents, protease inhibitors, acetylcholine esterase inhibitors, etc.

An object of the present disclosure is a Piezo agonist for use in the treatment of a neurodegenerative disease and/or neuroinflammatory disease, or a condition or disorder associated with a neurodegenerative disease and/or neuroinflammatory disease. Said neurodegenerative and neuroinflammatory diseases are evidenced with abnormal proinflammatory microglial activation.

An object of the present disclosure is a Piezo agonist for use in the microglia function modulation.

An object of the present disclosure is a method of treating a neurodegenerative disease and/or neuroinflammatory disease, or a condition or disorder associated with a neurodegenerative disease and/or neuroinflammatory disease, said method comprising administrating a Piezo agonist to a subject in need thereof, wherein the agonist is used for activating Piezo.

Another object of the invention is a method of treating conditions or disorders such as neurodegenerative diseases or a condition or disorder associated with said neurodegenerative disease, said method comprising administrating Piezo agonist to a subject in need thereof, wherein the agonist is used for activating Piezo. Another object of the invention is a method of treating disease states associated with, or at risk of, cerebral amyloidosis, said method comprising administrating Piezo agonist to a subject in need thereof.

Still another object of the invention is a method for determining a risk or risks associated with development or presence of a neurodegenerative disease and/or neuroinflammatory disease, or a condition or disorder associated with said neurodegenerative disease and/or neuroinflammatory disease in a human subject, comprising the steps of:

a. providing a test sample and a control sample;

b. measuring baseline fluorescence intensity of the test sample and the control sample;

c. adding Piezo agonist to said test sample;

d. measuring fluorescence intensity of the test sample and the control sample;

e. determining the difference of the fluorescence intensity of the test sample and the control sample,

wherein the reduced fluorescence intensity of the test sample in step e as compared to the fluorescence intensity of the control sample is indicative of reduced Piezo receptor activity in said test sample and of risk for development or presence of a neurodegenerative disease and/or neuroinflammatory disease, or a condition or disorder associated with said neurodegenerative disease and/or neuroinflammatory disease in said human subject.

A further object of the invention is a use of the Piezo agonist for treating at least one condition selected from the list consisting of Alzheimer's disease, Parkinson's disease, stroke, head trauma(s), cerebral amyloid angiopathies, spongiform encephalopathies, cerebral amyloid disease, and scrapie, wherein the agonist is used for activating Piezo. Said conditions are evidenced with abnormal proinflammatory microglial activation.

Still a further object of the invention is a method of modulating microglia function in a cell comprising contacting the cell with the Piezo agonist.

### BRIEF DESCRIPTION OF THE DRAWINGS

FIGS. 1A-1H show levels of Piezo receptors expression and their functional role in microglial cells. Corresponding relative gene expression levels of Piezo 1 (FIG. 1A) and Piezo 2 (FIG. 1B) shown as a fold change and normalized to trigeminal neurons Piezo expression each sample done in m=3 biological replicates in independent experiments n=4. The expression of piezol in human induced pluripotent stem cell (hIPSC)-derived microglia (FIG. 1C) and in human SV40 microglial cell line (FIG. 1D). The hIPSC-derived microglia respond to piezol specific agonist Yoda1 by eliciting Ca²⁺-responses (FIG. 1 E). Surprisingly, preincubation with Aβ blocks the Yoda1-induced Ca²⁺-responses suggesting that Aβ specifically binds and prevents the proper function of piezol (FIG. 1E). Quantification of Yoda1 elicited response and the blockage by Aβ and piezol inhibitor gadolinium (GdCI) in hIPS-derived microglia (FIG. 1F) and in SV-40 cells (FIG. 1G). Aβ reduced microglia motility when followed over a 2-day period (FIG. 1H). All data are presented as mean+/−SEM with *p<0.05, **p<0.01, ***p<0.001 as analyzed by t test or two-way ANOVA followed by Bonferroni post hoc test.

FIG. 2A illustrates that activation of Piezo using Yoda1 decreases microglial secretion of pro-inflammatory cytokines. Yoda1 at 20 μM concentration (Y20) prevented the LPS induced secretion of pro-inflammatory IL-6, IL-8, MCP1 and increases the secretion of anti-inflammatory IL-10 (FIG. 2). hIPSC-derived microglia were used, n=4 per group, One-way Anova followed by Bonferroni posthoc, **p<0.01; ***p<0.001.

FIG. 2B illustrates that activation of Piezo using Yoda1 prevents microglial cell death as analysed by cytotoxicity assay. Example curves of the cytotoxicity dye uptake by microglia as measured over 72-hour period. Quantification of the dye uptake at 60-hour time point reveals that 2-20 μM Yoda1 (Y2-Y20) protects hiPSC-microglia from cell death upon time. n=3 experiments. Positive control 200 μM MPP+.

FIG. 3 illustrates that Yoda1 treatment lead to increase in Iba1 immunopositivity and decrease in Aβ deposits. FIG. 3A shows the quantification of Iba1 staining in the cortex and hippocampus. The representative images depict typical examples of i) control, ii) Yoda1 and iii GdCI treated hippocampi. FIG. 3B shows the quantification of WO2 staining in the cortex and hippocampus. Representative images of WO2 staining in the hippocampus of i) control, ii) Yoda1 and iii GdCI treated animals. FIG. 3C high magnification images of i) control and ii) Yoda1 treated animals demonstrate how Iba1 positive microglia (in green) are recruited around the Aβ deposits (in red). Yoda1 treated mice (ii) show increased number of Iba1 positive cells around the Aβ deposits compared to vehicle treated controls (i). All data are presented as mean+/−SEM with *p<0.05, **p<0.01, ***p<0.001 as analyzed by t test or two-way ANOVA followed by Bonferroni post hoc test.

FIG. 4 shows that Piezo1 is expressed in developing neurons and peaks at 3 days post culture (FIG. 4A). The growth of neuronal processes was measured by using live incucyte imaging. FIG. 4B shows typical examples of vehicle and Yoda1 treated neurons at the time of plating (0 h) and at 6 days in vitro (DIV6). Quantification of the incucyte images taken over time period of 150 hours show that when plated at equal density, vehicle treated cells formed neurites as expected, however, Yoda1 treated cells showed more branching points (FIG. 4C) and extended neurite length (FIG. 4D) compared to vehicle treated cells. Quantification of branch points (C) and Neurite length (D). VEH=vehicle; Yoda=Yoda1. All data are presented as mean+/−SEM with *p<0.05, ***p<0.001 as analyzed by t test or two-way ANOVA followed by Bonferroni post hoc test.

FIGS. 5A-5E illustrate that Yoda1 prevents hypoxia-induced apoptosis. Yoda1 treatment is able to almost completely block hypoxia-induced early (FIG. 5A) and late (FIG. 5B) apoptosis of N2A cells modeling neurons. Veh h =vehicle hypoxia and Yoda h =Yoda. Yoda1 was administered immediately after stroke and thereafter once a day during three consecutive days. Lesion size was measured with MRI at 1 DPI and 3 DPI. The Yoda1 treated mice showed significantly smaller lesion size at both timepoints (FIG. 5C). Representative MRI images from vehicle (upper images) and Yoda1 treated mice (lower images), respectively (FIG. 5D). Lesions are shown in white. Yoda1 treated ischemic mice show no apparent deficits in their ability to sense adhesive patches post stroke (FIG. 5E). All data are presented as mean+/−SEM with *p<0.05,**p<0.01, ***p<0.001 as analyzed by t test or two-way ANOVA followed by Bonferroni post hoc test.

FIGS. 6A-6E show results of testing of red blood cells (RBC) obtained from SXFAD mice in the early stage of AD pathology. In FIG. 6: 1st time point, mice 13-15 w (FIG. 6A), 2nd time point, mice 15-17 w (FIG. 6B), 3rd time point, mice 17-19 w (FIG. 6C), 4th time point, mice 19-21 w (FIG. 6D) and 5th time point, mice 21-23 w (FIG. 6E).

### DETAILED DESCRIPTION OF THE DISCLOSURE

Alzheimer's disease (AD) is characterized by loss of neuronal function in the central nervous system (CNS). This loss of function occurs predominantly around senile plaques, which mainly consist of amyloid-beta deposits. The exact mechanism by which neuronal death and loss of function occur is currently not well understood.

Currently therapies are focused on dissolving the beta amyloid deposits and this approach has not been very successful. One approach is to use cholera toxin-B covalently linked to myelin basic protein. Therapies based on anti-amyloid beta plaque antibodies (e.g. Bapineuzumab) jointly developed by Johnson & Johnson and Pfizer have failed in Phase III clinicals. In 2014, another beta amyloid plaque targeted antibody (Crenezumab) developed by Roche/Genentech failed to meet its phase II objectives. Thus, there is real need for developing new targets for preventing or reversing loss of neuronal function due Alzheimer.

Stroke, the loss of brain function due to disturbed blood flow to the brain, causes a massive burden on both the lives of affected individuals and the economy. The number of people suffering from stroke each year is estimated to be 15 million. Five million people die annually from stroke, while another 5 million patients are left permanently disabled. Given the ageing of the populations, it is expected that the incidence of stroke will continue to escalate in the future. Ischemic stroke, which represents more than 80% of all strokes, occurs when cerebral arteries are occluded, and is often caused by thromboembolism. Brain cells suffer from lack of oxygen and nutrients and begin to die within minutes after the onset. Damage and death of neurons leads to paralysis, loss of speech, vision, memory or coordination, and in the most severe cases, death. Despite extensive research, the only clinically available treatment for stroke is thrombolysis, in which the time window for treatment is only a few hours from the beginning of symptoms. More effective therapeutic approaches are urgently needed. The therapeutic effect of the majority of conventional medications aims to halt the acute phase of stroke, which starts at the onset of restricted blood flow to tissues and lasts for a few hours. Indeed, many treatments lose their effect if administration of the medication is delayed by a few hours. This creates the lack of a realistic, effective time window for treatment of patients. In the present invention, the protection from stroke is mediated by microglia.

Parkinson's disease is a long-term degenerative disorder of the central nervous system that mainly affects the motor system. There is speculation of several mechanisms by which the brain cells could be lost. One mechanism consists of an abnormal accumulation of the protein alpha-synuclein bound to ubiquitin in the damaged cells. Other cell-death mechanisms include proteasomal and lysosomal system dysfunction and reduced mitochondrial activity.

Head trauma can be as mild as a bump, bruise (contusion), or cut on the head, or can be moderate to severe in nature due to a concussion, deep cut or open wound, fractured skull bone(s), or from internal bleeding and damage to the brain. A head trauma is a broad term that describes a vast array of injuries that occur to the scalp, skull, brain, and underlying tissue and blood vessels in the head. Head injuries are also commonly referred to as brain injury, or traumatic brain injury (TBI), depending on the extent of the head trauma.

Cerebral amyloid angiopathy (CAA), also known as congophilic angiopathy, is a form of angiopathy in which amyloid deposits form in the walls of the blood vessels of the central nervous system. The amyloid material is only found in the brain and as such the disease is not related to other forms of amyloidosis. CAA is defined by the deposition of Aβ in the leptomeningal and cerebral vessel walls. The reason for increased deposition of Aβ in sporadic CAA is still unclear with both increased production of the peptide and abnormal clearance having been proposed as potential causes.

Transmissible spongiform encephalopathies (TSEs) are a group of progressive, invariably fatal, conditions that are associated with prions and affect the brain (encephalopathies) and nervous system of many animals, including humans, cattle, and sheep. According to the most widespread hypothesis, they are transmitted by prions, though some other data suggest an involvement of a Spiroplasma infection. TSEs of humans include Creutzfeldt-Jakob disease—which has four main forms, the sporadic (sCJD), the hereditary/familiar (fCJD), the iatrogenic (iCJD) and the variant form (vCJD)—Gerstmann-Sträussler-Scheinker syndrome, fatal familial insomnia, kuru, and the recently discovered variably protease-sensitive prionopathy. These conditions form a spectrum of diseases with overlapping signs and symptoms. TSEs in non-human mammals include scrapie in sheep, bovine spongiform encephalopathy (BSE)—popularly known as “mad cow's disease”—in cattle and chronic wasting disease (CWD)—also known as ‘zombie deer disease’—in deer and elk.

An active agent binding to Piezo can be administered in form of a pharmaceutical composition, such as an antibody, nucleotide, small molecule or an activating binding compound for the ion channel function.

Preferably, the active agent is Piezo agonist. More preferably said agonist is Yoda1 having the formula I

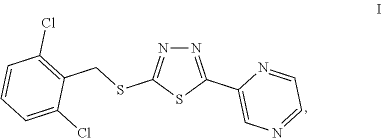 [SMILES: Clc1cccc(Cl)c1CSc1nnc(-c2cnccn2)s1]

Jedi1 having the formula II

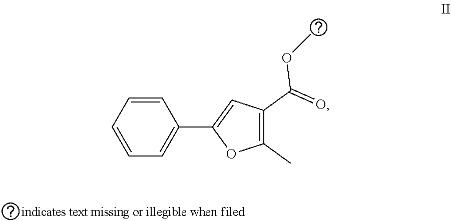 [SMILES: COC(=O)c1cc(-c2ccccc2)oc1C]

or Jedi2 having the formula III

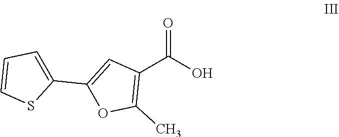

or a functional analog or functional analogs thereof. The term “functional analog” as used herein refers to chemical compounds that have similar physical, chemical, biochemical, or pharmacological properties. The term “agonist” refers to those that can positively influence the process, e.g, can activate or stimulate the process, and include chemical, biochemical, cellular or physiological processes. It should be understood that it includes but is not limited to these. A “Piezo agonist” is understood to refer to a molecule capable of activating a Piezo receptor. A piezo agonist can bind to a Piezo receptor. Examples of Piezo agonists are Yoda1, Jedi1 and Jedi2, and functional analogs thereof. Most preferably the agonist is Yoda1. The amount of agonist used to activate Piezo is between 1 nM-50 μM. Preferably, the amount can be 100 nM, 200 nM, 300 nM, 400 nM, 500 nM, 600 nM, 700 nM, 800 nM, 900 nM, 100nm-1 μM, 500 nM-1 μM, 1 μM-25 μM, 1 μM-20 μM, 1 μM-15 μM, 1 μM-10 μM, 1 μM-5 μM, 5 μM-10 μM, 5 μM-15 μM, 10 μM-15 μM, 10 μM-20 μM, 10 μM-25 μM, 10 μM-30 μM, 15 μM-30 μM, 20 μM-30 μM, 25 μM-30 μM, 30 μM-40 μM, 30 μM-50 μM, 40 μM-50 μM, 45 μM-50 μM, 1 μM, 2 μM, 3 μM, 4 μM, 5 μM, 6 μM, 7 μM, 8 μM, 9 μM, 10 μM, 11 μM, 12 μM, 13 μM, 14 μM, 15 μM, 16 μM, 17 μM, 18 μM, 19 μM, 20 μM, 21 μM, 22 μM, 23 μM, 24 μM, 25 μM, 26 μM, 27 μM, 28 μM, 29 μM, 30 μM, 40 μM or 50 μM. More preferably, the amount is 10 μM-50 μM.

An “effective amount” is an amount of the compound(s) or the pharmaceutical composition as described herein that induces on the expression and/or abundance of the ion channel or induces the activity of the ion channel. The amount alleviates symptoms as found for neurodegenerative disease and/or neuroinflammatory disease or a condition or disorder associated with said neurodegenerative disease and/or neuroinflammatory disease. Alleviating is meant to include, e.g., preventing, treating, reducing the symptoms of, or curing the disease (such as AD, Parkinson's disease, stroke, head trauma(s), cerebral amyloid angiopathies, spongiform encephalopathies and scrapie) or condition (e.g. plaque formation).

In one embodiment, a therapeutically effective dosage of the compound, such as Piezo agonist, disclosed herein from about 0.1 mg to about 2,000 mg per day. The pharmaceutical compositions should provide a dosage of from about 0.1 mg to about 2000 mg of the compound. In a special embodiment, pharmaceutical dosage unit forms are prepared to provide from about 1 mg to about 2,000 mg, about 10 mg to about 1,000 mg, about 20 mg to about 500 mg, or about 25 mg to about 250 mg of the active ingredient or a combination of essential ingredients per dosage unit form. In a special embodiment, pharmaceutical dosage unit forms are prepared to provide about 10 mg, 20 mg, 25 mg, 50 mg, 100 mg, 250 mg, 500 mg, 1000 mg or 2000 mg of the active ingredient.

One object of the invention is a Piezo agonist for use in the treatment of a neurodegenerative and/or neuroinflammatory disease, or a condition or disorder associated with a neurodegenerative disease and/or neuroinflammatory disease.

An aspect of the invention is a Piezo agonist for use in preventing a neurodegenerative and/or neuroinflammatory disease, or a condition or disorder associated with a neurodegenerative disease and/or neuroinflammatory disease.

In an embodiment a disease or condition or disorder to be treated and/or prevented is an inflammatory disease or condition or disorder, preferably a neuroinflammatory disease or condition or disorder.

In an embodiment the Piezo agonist is for use in the treatment wherein the Piezo is Piezo1 or Piezo2. In an embodiment the Piezo agonist is for use in the treatment wherein the Piezo is from a mouse or a human being. In an embodiment the Piezo agonist is for use in the treatment wherein the Piezo agonist is used for activating Piezo. In an embodiment the Piezo agonist is for use in the treatment wherein the Piezo agonist is selected from the group consisting of Yoda1, Jedi1, Jedi2, and functional analogs thereof. In a preferred embodiment for use in the treatment the Piezo agonist is Yoda1.

One object of the invention is a Piezo agonist for use in the microglia function modulation.ln an embodiment the Piezo agonist is for use in the treatment wherein the microglia function to be modulated is motility, phagocytosis and/or cytokine release.

According to one embodiment the modulated microglia function is its phagocytosis and/or cytokine release. Activation of PiezoRs1 leads to decrease in microglial cytokine production and enhances the phagocytic capacity of the microglia. Cytokines that relate to microglia are for example proinflammatory cytokines e.g. IL-1β, IFNγ, IL-6, IL-2 and TNFa but can also include a variety of other cytokines. Representative cytokines include, but are not limited to, the group consisting of interleukin-1a (IL-1a), interleukin-3 (IL-3), interleukin-2 (IL-2), interleukin-4 (IL-4), interleukin-5 (IL-5), interleukin-6 (IL-6), interleukin-7 (IL-7), interleukin-8 (IL-8/CXCL8), interleukin-10 (IL-10), interleukin-12 (IL-12), interleukin-13 (IL-13), interleukin-15 (IL-15), interleukin-17 (IL-17), interleukin-18 (IL-18), tumor necrosis factor-α (TNF-α), interferon-β(INF-β), interferon-α(INF-α), interferon-γ (INF-γ), granulocyte monocyte colony stimulating factor (GM-CSF), granulocyte colony stimulating factor (G-CSF), monocyte chemoattractant protein-1 (MCP-1/CCL2), macrophage inflammatory protein 1-α (MIP-1α/CCL3), macrophage inflammatory protein-13 (MIP-13/CCL4), RANTES (CCL5), Eotaxin (CCL11), variable endothelial growth factor (VEGF), endothelial growth factor (EGF), and fibroblast growth factor (FGF).

In an embodiment the Piezo agonist is for use in the treatment wherein amyloid-β accumulation is inhibited.

In an embodiment the Piezo agonist is for use in the treatment wherein the load of amyloid-β plaques is reduced.

In an embodiment the Piezo agonist is for use in the treatment wherein the Piezo agonist is used in combination with at least one molecule selected from the group consisting of: arachidonoylethanolamide, 2-arachidonoylglycerol, palmitoylethanolamide, oleoylethanolamide, and linoleoyl ethanolamide.

In an embodiment the Piezo agonist is for use in the, wherein the neurodegenerative and/or neuroinflammatory disease, or a condition or disorder associated with a neurodegenerative disease and/or neuroinflammatory disease is selected from the group consisting of Alzheimer's disease, stroke, Parkinson's disease, head trauma(s), cerebral amyloid angiopathies, spongiform encephalopathies, cerebral amyloid disease, and scrapie. Said neurodegenerative and/or neuroinflammatory diseases are evidenced with abnormal proinflammatory microglial activation.

In an embodiment the Piezo agonist is for use in the treatment in pharmaceutical compositions for the preventive or therapeutic treatment of a neurodegenerative disease and/or neuroinflammatory disease, or a condition or disorder associated with neurodegenerative disease and/or neuroinflammatory disease.

One object of the invention is a piezo agonist for treating neurodegenerative disease or a condition or disorder associated with said neurodegenerative disease. More preferably the neurodegenerative disease or a condition or disorder associated with said neurodegenerative disease includes at least one of the following: Alzheimer's disease, Parkinson's disease, stroke, head trauma(s), cerebral amyloid angiopathies, spongiform encephalopathies and scrapie. Said neurodegenerative diseases or conditions or disorders are evidenced with abnormal proinflammatory microglial activation.

Preferably, a patient is a human being. Treating is meant to include, e.g., preventing, treating, reducing the symptoms of, or curing the disease or condition, i.e. neurodegenerative disease or a condition or disorder associated with said neurodegenerative disease, which is evidenced with abnormal proinflammatory microglial activation.

Preferred is a method according to the present invention, wherein said neurodegenerative disease or a condition or disorder associated with said neurodegenerative disease is a cerebral amyloidogenic disease and is selected from AD, Parkinson's disease, stroke, head trauma(s), cerebral amyloid angiopathies, spongiform encephalopathies and scrapie.

The invention also includes a method for treating a subject at risk for a neurodegenerative disease, wherein a therapeutically effective amount of a modulator as above is provided. Being at risk for the disease can result from, e.g., a family history of the disease, a genotype which predisposes to the disease, or phenotypic symptoms which predispose to the disease. A further aspect of the present invention is the use of a modulator of the expression and/or the biological activity of the ion channel for the manufacture of a pharmaceutical composition for treating or preventing a neurodegenerative disease or a condition or disorder associated with said neurodegenerative disease. Preferably, said modulator is an activator of the expression and/or biological activity of the ion channel as described herein.

The present invention also provides a pharmaceutical composition comprising regulator used for activating or inhibiting Piezo, such as Piezo activator Yoda1, Jedi1 or Jedi2, or functional analogs thereof. According to the specific examples of the present invention, the pharmaceutical composition can further comprise pharmaceutically acceptable excipient, carrier, adjuvant, solvent and a combination thereof.

The present invention provides a method of treating, preventing or ameliorating a disease or disorder, comprising administrating a safe and effective amount of a combination of drugs containing compounds and one or more therapeutic active agents. Among them, the combination of drugs comprises one or more additional drugs for treatment of Piezo-related disease.

It will also be appreciated that certain of the compounds of the present invention can exist in free form for treatment, or where appropriate, as a pharmaceutically acceptable derivative or a prodrug thereof. A pharmaceutically acceptable derivative includes pharmaceutically acceptable salts, esters, salts of such esters, or any other adduct or derivative which upon administration to a patient in need thereof is capable of providing, directly or indirectly, a compound as otherwise described herein, or a metabolite or residue thereof.

When the pharmaceutical compositions of the present invention also contain one or more other active ingredients, in addition to a compound of the present invention. “Pharmaceutically acceptable excipient” as used herein means a pharmaceutically acceptable material, composition or vehicle involved in giving form or consistency to the pharmaceutical composition. Suitable pharmaceutically acceptable excipients will vary depending upon the particular dosage form chosen. In addition, suitable pharmaceutically acceptable excipients may be chosen for a particular function that they may serve in the composition. Suitable pharmaceutically acceptable excipients include the following types of excipients: diluents, fillers, binders, disintegrants, lubricants, glidants, granulating agents, coating agents, wetting agents, solvents, co-solvents, suspending agents, emulsifiers, sweetners, flavoring agents, flavor masking agents, coloring agents, anticaking agents, humectants, chelating agents, plasticizers, viscosity increasing agents, antioxidants, preservatives, stabilizers, surfactants, and buffering agents. The skilled artisan will appreciate that certain pharmaceutically acceptable excipients may serve more than one function and may serve alternative functions depending on how much of the excipient is present in the formulation and what other ingredients are present in the formulation.

The pharmaceutical composition contains the compound disclosed herein and pharmaceutically acceptable excipient, carrier, adjuvant, vehicle or a combination thereof, the method comprises mixing various ingredients.

The molecules, object of the present invention, can be employed, alone or in association with other selected therapeutic agents, for preparation of pharmaceutical compositions useful for specific therapeutic purposes. The selected therapeutic agents to be used in combination with the molecules object of the present invention, can be chosen among: anti-oxidants, protease inhibitors, acetylcholine esterase inhibitors, anti-convulsivants, neuroleptics, atypical neuroleptics, anti-depressants, dopamine-agonists, GABA-agonists, drugs for memory improvement, anti-inflammatory drugs, pain-killers (eg. opiods, salycilates, pyrazolics, indolics, anthranilics, aryl-propyonics, aryl-acetics, oxicams, pyranocarboxilics, glucocorticoids, cox2 inhibitors, and acetaminophen).

The compound of the invention will typically be formulated into a dosage form adapted for administration to the patient by the desired route of administration. For example, dosage forms include those adapted for oral administration such as tablets, capsules, caplets, pills, troches, powders, syrups, elixirs, suspensions, solutions, emulsions, sachets, and cachets parenteral administration such as sterile solutions, suspensions, and powders for reconstitution transdermal administration such as transdermal patches; rectal administration such as suppositories inhalation such as aerosols, solutions, and dry powders and topical administration such as creams, ointments, lotions, solutions, pastes, sprays, foams, and gels.

The pharmaceutical compositions provided herein may be administered parenterally by injection, infusion, or implantation, for local or systemic administration. Parenteral administration, as used herein, include intravenous, intraarterial, intraperitoneal, intrathecal, intraventricular, intraurethral, intrasternal, intracranial, intramuscular, intrasynovial, and subcutaneous administration. In addition, administration can be oral or intravenous.

The pharmaceutical compositions intended for parenteral administration may include one or more pharmaceutically acceptable carriers and excipients, including, but not limited to, aqueous vehicles, water-miscible vehicles, non-aqueous vehicles, antimicrobial agents or preservatives against the growth of microorganisms, stabilizers, solubility enhancers, isotonic agents, buffering agents, antioxidants, local anesthetics, suspending and dispersing agents, wetting or emulsifying agents, complexing agents, sequestering or chelating agents, cryoprotectants, lyoprotectants, thickening agents, pH adjusting agents, and inert gases.

The pharmaceutical compositions provided herein may be formulated for single or multiple dosage administration. The single dosage formulations are packaged in an ampoule, a vial, or a syringe. The multiple dosage parenteral formulations must contain an antimicrobial agent at bacteriostatic or fungistatic concentrations. All parenteral formulations must be sterile, as known and practiced in the art.

In one embodiment, the compound of the invention or the pharmaceutical composition thereof may be administered once or according to a dosing regimen wherein a number of doses are administered at varying intervals of time for a given period of time. For example, doses may be administered one, two, three, or four times per day. Generally, dosage levels of between 0.0001 to 10 mg/kg of body weight daily are administered to the patient to obtain effective modulation of Piezo.

The compounds of the present invention may be administered either simultaneously with, or before or after, one or more other therapeutic agents. The compounds of the present invention may be administered separately, by the same or different route of administration, or together in the same pharmaceutical composition as the other agents.

Piezo channels can be employed not only for reduction of plaques in dementia states but also as a biomarker of susceptibility to Alzheimer's Disease in the pre-symptomatic period.

This approach is based on the vast expression of Piezo channels in the red blood cells (RBC) and also blood mononuclear cells. Due to known inhibitory action of Aβ on Piezol protein, the latter can reduce or lost the function in the early stages of AD. A little invasive approach to get a small amount of blood sample with subsequent flow cytometry testing the function of Piezo1 channels activated by Yoda1 can be used for the wide screening for the predisposition to the AD in middle age subjects when the prophylaxis of the subsequent dementia is the most effective.

One object of the present invention is a method for determining a risk associated with development or presence of a neurodegenerative disease and/or neuroinflammatory disease, or a condition or disorder associated with said neurodegenerative disease and/or neuroinflammatory disease in a human subject, comprising the steps of:

a. providing a test sample and a control sample;

b. measuring baseline fluorescence intensity of the sample and the control sample;

c. adding Piezo agonist to said test sample;

d. measuring fluorescence intensity of the test sample and the control sample;

e. determining mean fluorescence intensity of the test sample,

wherein the reduced fluorescence intensity of the test sample in step e as compared to the fluorescence intensity of the control sample is indicative of reduced Piezo receptor activity in said test sample and of risk for development or presence of a neurodegenerative disease and/or neuroinflammatory disease, or a condition or disorder associated with said neurodegenerative disease and/or neuroinflammatory disease in said human subject.

One object of the present invention is a method for determining risks associated with development or presence of a neurodegenerative disease or a condition or disorder associated with said neurodegenerative disease in a human subject, comprising the steps of:

a. providing a test sample;

b. adding calcium indicator to said sample;

c. measuring baseline fluorescence intensity of the sample;

d. adding Piezo agonist to said test sample;

e. measuring fluorescence intensity of the test sample;

f. adding calcium ionophore to said test sample;

g. measuring fluorescence intensity of the test sample; and

h. determining mean fluorescence intensity of the test sample,

wherein the reduced fluorescence intensity of the test sample in step e as compared to the fluorescence intensity of step c is indicative of reduced Piezo receptor activity in said test sample and of risk for development or presence of neurodegenerative disease or a condition or disorder associated with said neurodegenerative disease in said human subject.

One object of the present invention is a method for determining risks associated with development or presence of a neurodegenerative disease or a condition or disorder associated with said neurodegenerative disease in a human subject, comprising the steps of:

a. providing a test sample and a control sample;

b. adding calcium indicator to said test sample and/or the control sample;

c. measuring baseline fluorescence intensity of the test sample and the control sample;

d. adding Piezo agonist to said test sample;

e. measuring fluorescence intensity of the test sample and the control sample;

f. adding calcium ionophore to said test sample and/or the control sample;

g. measuring fluorescence intensity of the test sample and the control sample; and

h. determining mean fluorescence intensity of the test sample,

wherein the reduced fluorescence intensity of the test sample in step e as compared to the fluorescence intensity of the control sample is indicative of reduced Piezo receptor activity in said test sample and of risk for development or presence of neurodegenerative disease or a condition or disorder associated with said neurodegenerative disease in said human subject.Calcium indicators, such as synthetic Ca²⁺dyes Fluo-4, Fluo-5F, Fluo-4FF, Rhod-2, X-Rhod-5F, Oregon Green 488 BAPTA-6F, Fluo-8, Fluo-8 high affinity, Fluo-8 low affinity, Oregon Green BAPTA-1, Cal-520, Rhod-4, Asante Calcium Red, and X-Rhod-1 as well as genetically-encoded Ca²⁺-indicators such as GCaMP6-slow, -medium and -fast variants can be used in the invention. Preferably, the indicator is Fluo-4.

Calcium ionophore is used as positive control in the test. Calcium ionophore releases all Ca resources (i.e. is a Ca-uncoupler) and indicates that cells are alive and functional. Calcium ionophores, such as ionomycin, calcimycin (A23187) or Calcium ionophore V (K23E1) can be used in the present invention. Preferably, the ionophore is ionomycin.

The detected reduction of at least 10% in Ca²⁺ influx in a test sample as compared to the baseline measurement is indicative of reduced Piezo receptor activity in said test sample and suggests increased amyloid-β accumulation in the human subject. Preferably, the Ca²⁺ influx reduction can be 10-100%, 10-90%, 10-85%, 10-80%, 10-75%, 10-70%, 10-65%, 10-60%, 10-55%, 10-50%, 10-45%, 10-40%, 10-35%, 10-30%, 10-25%, 20-100%, 20-90%, 20-85%, 20-80%, 20-75%, 20-70%, 20-65%, 20-60%, 20-55%, 20-50%, 20-45%, 20-40%, 20-35%, 30-100%, 30-90%, 30-85%, 30-80%, 30-75%, 30-70%, 30-65%, 30-60%, 30-55%, 30-50%, 30-45%, 40-100%, 40-90%, 40-85%, 40-80%, 40-75%, 40-70%, 40-65%, 40-60%, 50-100%, 50-90%, 50-85%, 50-80%, 50-75%, 50-70%, 60-100%, 60-90%, 60-85%, 60-80%, 60-75%, 70-100%, 80-100% or 90-100%.

The most preferred biomarkers according to the present invention are Piezo1 and Piezo2, or a combination thereof.

In general, the molecular biomarker may be detected in body fluids e.g., blood and blood plasma, lymph, liquid and/or urine. In preferred embodiments, the molecular biomarker may be detected in the blood, especially in red blood cells or blood mononuclear cells. The biomarker can be easily examined. For the purposes of the present invention, the biomarker protein (Piezo) activity is decreased in quantity when the subject has a condition associated with amyloid-beta accumulation, or a risk for development of neurodegenerative disease or has a neurodegenerative disease and increased in response to the treatment with a Piezo agonist.

The native cannabinoid receptor ligands aka “endocannabinoids” are classically represented by arachidonylethanolamide (anandamide, AEA) and 2-arachidonoylglycerol (2AG). Tissue levels of endocannabinoids are maintained by the balance between biosynthesis (e.g., phospholipase D and diacylglycerol lipase-dependent and other pathways), cellular uptake and degradation by enzymes principally, but not limited to fatty acid amide hydrolase (FAAH) and/or monoacylglycerol lipases (MAGL). Since the discovery of CB₁ and CB2GPCRs such as GPR₁₈, GPR55, GPR₁₁₉ and the TRPs have been recognized as members of the cannabinoid family. (CB=cannabinoids; GPCR=G-protein coupled receptors and TRP =transient receptor potential).

Endocannabinoids (MAGL and FAAH) have been shown to have drastic anti-inflammatory effects. This is based on the accumulation of endogenous cannabinoids, such as 2-AG and AEA limiting the overuse and psychotrophic which can happen with exogenously applied cannabinoids. Moreover, such inhibition limits the generation of downstream endocannan products, such as arachidonic acid and its derivatives which exert strong pro-inflammatory action. Combinatory treatment with endocannabinoids and piezo agonist is likely to yield a dual synergistic protective effect.

The combinatorial approach of the present inventors to treat plaques would benefit primarily from the combination of Piezo agonists with the potent agents accumulating endocannabinoids 2-AG and AEA, The most efficient way to accumulate 2-AG and AEA is to block enzymes MAGL and FAAH with currently available super potent compounds JJKK-048 and JZP-372A, respectively. This novel approach fits with recent data showing that 2-AG exerts protective actions in CNS injury models modulating microglia towards an anti-inflammatory state via CB2 receptor and reducing expression of the pro-inflammatory cytokines.

Embodiments of the invention may include one or more molecules selected from the group consisting of mammalian cannabinoids. Preferred embodiments of the invention might be formulated to result in increased presence or activity of at least one active ingredient comprises at least one molecule selected from the group consisting of: arachidonoylethanolamide (AEA), 2-arachidonoylglycerol (2AG), palmitoylethanolamide (PEA), oleoylethanolamide (OEA) and linoleoyl ethanolamide (LEA). PEA, OEA and LEA are N-Acylethanolamines. One or more embodiments of the present invention may have at least one active ingredient selected from the group consisting of: URB597, URB937, AM374, ARN2508, BIA 10-2474, BMS-469908, CAY-10402, JNJ-245, JNJ-1661010, JNJ-28833155, JNJ-40413269, JNJ-42119779, JNJ-42165279, LY-2183240, cannabidiol, MK-3168, MK-4409, MM-433593, OL-92, OL-135, PF-622, PF-750, PF-3845, PF-04457845, PF-04862853, RN-450, SA-47, SA-73, SSR-411298, ST-4068, TK-25, URB524, URB597 (KDS-4103), URB694, URB937, VER-156084, V-158866, AM3506, AM6701, CAY10435, CAY10499, IDFP, JJKK-048, JNJ-40355003, JNJ-5003, JW618, JW651, JZL184, JZL195, JZP-372A, KML29, MAFP, MJN110,ML30, N-arachidonoyl maleimide, OL-135, OL92, PF-04457845, SA-57, ST4070, URB880, URB937, indomethacin, MK-886, resveratrol, cis-resveratrol, aspirin, COX-1 inhibitor II, loganin, tenidap, SC560, FR 122047 hydrochloride, valeryl salicylate, FR122047 hydrate, ibuprofen, TFAP, 6-methoxy-2-naphthylacetic acid, meloxicam, APHS, etodolac, meloxicam, meloxicam sodium salt, N-(4-acetamidophenyl)indomethacin amide, N-(2-phenylethyl)indomethacin amide, N-(3-pyridyl)indomethacin amide, indomethacin heptyl ester, SC236, sulinac, sulindac sulfide, pravadoline, naproxen, naproxen sodium salt, meclofenamate sodium, ibupropfen, S-ibuprofen, piroxicam, ketoprofen, S-ketoprofen, R-ibuprofen, ebselen, ETYA, diclofenac, diclofenac diethylamine, flurbiprofen, fexofenadine, Pterostilbene, Pterocarpus marsupium, 9,12-octadecadiynoic acid, Ketorolac (tromethamine salt), NO-indomethacin, S-flurbiprofen, sedanolide, green tea extract (e.g., epicatechin), licofelone, lornoxicam, rac ibuprofen-d3, ampirxicam, zaltoprofen, 7-(trifluoromethyl)1 H-indole-2,3-dione, aceclofenac, acetylsalicylic acid-d4, S-ibuprofen lysinate, loxoprofen, CAY10589, ZU-6, isoicam, dipyrone, YS121 and MEG (mercaptoethylguanidine). Preferred embodiments may incorporate 2, 3, 4, 5, 6 or even more cannabinolic supportive compounds or enzymes.

Solvents for the purpose of the invention may not interfere with the biological activity of the solute. Examples of suitable solvents include, but are not limited to, water, methanol, ethanol, oleic acid and acetic acid or organic solvent, for example THF, DMF, dichloromethane (DCM), ethyl acetate (EtOAc) or acetonitrile. Preferably the solvent used is a pharmaceutically acceptable solvent. Examples of suitable pharmaceutically acceptable solvents include, without limitation, methyl cellulose, water, ethanol and acetic acid.

An aspect of the invention is a Piezo agonist for use in the treatment of a neurodegenerative and/or neuroinflammatory disease, or a condition or disorder associated with a neurodegenerative disease and/or neuroinflammatory disease. In an embodiment the Piezo agonist is for use in the treatment wherein the Piezo is Piezo1 or Piezo2. In an embodiment the Piezo agonist is for use in the treatment wherein the Piezo is from a mouse or a human being.

In an embodiment the Piezo agonist is for use in the treatment wherein the Piezo agonist is for modulation of microglia function. In a preferred embodiment the Piezo agonist is for use in the treatment wherein the microglia function to be modulated is motility, phagocytosis and/or cytokine release.

In an embodiment the Piezo agonist is for use in the treatment wherein the Piezo agonist is used for activating Piezo.

In an embodiment the Piezo agonist is for use in the treatment wherein the Piezo agonist is selected from the group consisting of Yoda1, Jedi1, Jedi2, and functional analogs thereof. In a preferred embodiment for use in the treatment the Piezo agonist is Yoda1.

In an embodiment the Piezo agonist is for use in the treatment wherein amyloid-β accumulation is inhibited.

In an embodiment the Piezo agonist is for use in the treatment wherein the load of amyloid-β plaques is reduced.

In an embodiment the Piezo agonist is for use in the treatment wherein the Piezo agonist is used in combination with at least one molecule selected from the group consisting of: arachidonoylethanolamide, 2-arachidonoylglycerol, palmitoylethanolamide, oleoylethanolamide, and linoleoyl ethanolamide.

In an embodiment the Piezo agonist is for use in the, wherein the neurodegenerative and/or neuroinflammatory disease, or a condition or disorder associated with a neurodegenerative disease and/or neuroinflammatory disease is selected from the group consisting of Alzheimer's disease, stroke, Parkinson's disease, head trauma(s), cerebral amyloid angiopathies, spongiform encephalopathies, cerebral amyloid disease, and scrapie. Said neurodegenerative or neuroinflammatory diseases are evidenced with abnormal proinflammatory microglial activation.

In an embodiment the Piezo agonist is for use in the treatment in pharmaceutical compositions for the preventive or therapeutic treatment of a neurodegenerative disease and/or neuroinflammatory disease, or a condition or disorder associated with neurodegenerative disease and/or neuroinflammatory disease.

In an embodiment a disease or condition or disorder to be treated and/or prevented is an inflammatory disease or condition or disorder, preferably a neuroinflammatory disease or condition or disorder.

An aspect of the invention is a method of treating a neurodegenerative and/or neuroinflammatory disease, or a condition or disorder associated with a neurodegenerative disease and/or neuroinflammatory disease, said method comprising administrating a Piezo agonist to a subject in need thereof, wherein the agonist is used for activating Piezo.

In an embodiment of the method the Piezo agonist is selected from the group consisting of Yoda1, Jedi1, Jedi2, and functional analogs thereof. In a preferred embodiment of the method the Piezo agonist is Yoda1.

In an embodiment of the method the Piezo agonist is used in combination with at least one molecule selected from the group consisting of: arachidonoylethanolamide, 2-arachidonoylglycerol, palmitoylethanolamide, oleoylethanolamide and linoleoyl ethanolamide.

An object of the present invention is a Piezo agonist for use in the microglia function modulation. In a more preferred embodiment, the Piezo itself is Piezo1 or Piezo2. In still more preferred embodiment Piezo is from mouse or human beings. According to one preferred embodiment, the agonist is used for activating Piezo. According to another preferred embodiment, the agonist is Yoda1, Jedi1, Jedi2, or functional analogs thereof. According to one preferred embodiment, the microglia function to be modulated is motility, phagocytosis and/or cytokine release. In a preferred embodiment, beta-amyloid accumulation is inhibited. In still another preferred embodiment, the load of beta-amyloid plaques is reduced.

According to one preferred embodiment, the Piezo agonist is for use in the treatment of conditions or disorders associated with cerebral amyloidogenic diseases. According to a still more preferred embodiment, when the Piezo agonist is for use in the treatment of neurodegenerative diseases or a condition or disorder associated with said neurodegenerative disease, Piezo agonist is used in combination with at least one molecule selected from the group consisting of: arachidonoylethanolamide (AEA), 2-arachidonoylglycerol (2-AG), palmitoylethanolamide (PEA), oleoylethanolamide (OEA) and linoleoyl ethanolamide (LEA). The Piezo agonist can also be used when the condition or disorder associated with neurodegenerative diseases is selected from the group consisting of Alzheimer's disease, Parkinson's disease, stroke, head trauma(s), cerebral amyloid angiopathies, spongiform encephalopathies and scrapie. Said neurodegenerative diseases are evidenced with abnormal proinflammatory microglial activation According to a preferred embodiment, the Piezo agonist is for use in pharmaceutical compositions for the preventive or therapeutic treatment of conditions or disorders associated with cerebral amyloidogenic diseases.

According to one embodiment is a method of treating conditions or disorders associated with neurodegenerative diseases, or neurodegenerative diseases as such, said method comprising administrating Piezo agonist to a subject in need thereof, wherein the agonist is used for activating Piezo. According to a preferred embodiment is a method of treating conditions or disorders, wherein Piezo agonist is used in combination with at least one molecule selected from the group consisting of: AEA, 2-AG, PEA, OEA and LEA.

Method of treating neurodegenerative disease or a condition or disorder associated with said neurodegenerative disease and/or neuroinflammatory disease including at least one of the following: Alzheimer's disease, Parkinson's disease, stroke, head trauma(s), cerebral amyloid angiopathies, spongiform encephalopathies, cerebral amyloid disease and scrapie, is also one preferred aspect of the invention.

A method for determining a risk associated with development or presence of a neurodegenerative disease and/or neuroinflammatory disease, or a condition or disorder associated with said neurodegenerative disease and/or neuroinflammatory disease in a human subject, comprising the steps of:

a. providing a test sample and a control sample;

b. measuring baseline fluorescence intensity of the test sample and the control sample;

c. adding Piezo agonist to said test sample;

d. measuring fluorescence intensity of the test sample and the control sample;

e. determining the difference of the fluorescence intensity of the test sample and the control sample,

wherein the reduced fluorescence intensity of the test sample in step e as compared to the fluorescence intensity of the control sample is indicative of reduced Piezo receptor activity in said test sample and of risk for development or presence of a neurodegenerative and/or neuroinflammatory disease, or a condition or disorder associated with said neurodegenerative disease and/or neuroinflammatory disease in said human subject is also an aspect of the invention.

A method for determining a risk or risks associated with development or presence of a neurodegenerative disease and/or neuroinflammatory disease, or a condition or disorder associated with said neurodegenerative disease and/or neuroinflammatory disease in a human subject, comprising the steps of:

a. providing a test sample;

b. adding calcium indicator to said sample;

c. measuring baseline fluorescence intensity of the sample;

d. adding Piezo agonist to said test sample;

e. measuring fluorescence intensity of the test sample;

f. adding calcium ionophore to said test sample;

g. measuring fluorescence intensity of the test sample; and

h. determining mean fluorescence intensity of the test sample,

wherein the reduced fluorescence intensity of the test sample in step e as compared to the fluorescence intensity of step c is indicative of reduced Piezo receptor activity in said test sample and presence or risk of neurodegenerative disease or disorder associated with said neurodegenerative disease in said human subject., is also an aspect of the invention. According to a preferred embodiment of the method, the sample used in the method consists of red blood cells, blood mononuclear cells, serum, plasma or whole blood. More preferably the sample consists of red blood cells or blood mononuclear cells.

In an embodiment of the method the neurodegenerative and/or neuroinflammatory disease, or a condition or disorder associated with said neurodegenerative disease and/or neuroinflammatory disease includes at least one of the following: Alzheimer's disease, Parkinson's disease, stroke, head trauma(s), cerebral amyloid angiopathies, spongiform encephalopathies, cerebral amyloid disease, and scrapie. Said neurodegenerative or neuroinflammatory diseases are evidenced with abnormal proinflammatory microglial activation.

In an embodiment of the method the condition, disease or disorder is, or is associated with at least one neurodegenerative disease and/or neuroinflammatory disease selected form the group consisting of: Alzheimer's disease, Parkinson's disease, stroke, head trauma(s), cerebral amyloid angiopathies, spongiform encephalopathies, cerebral amyloid disease, and scrapie.

Also use of the Piezo agonist for treating at least one condition selected from the list consisting of Alzheimer's disease, Parkinson's disease, stroke, head trauma(s), cerebral amyloid angiopathies, spongiform encephalopathies, cerebral amyloid disease and scrapie, wherein the agonist is used for activating Piezo, is an aspect of the invention.

A method of modulating microglia function in a microglial cell comprising contacting the cell with the Piezo agonist is a further aspect of the invention.

### EXAMPLES

Materials and Methods

qRT-PCR

To demonstrate expression levels of Piezo receptors (Piezo1 Rs and Piezo2) in the different microglia cell types the present inventors used qPCR (TaqMan Kit, Tsher

Scientific). Nucleic acids concentrations were measured at 260 nm. Purity of the RNA extracted was determined as the 260 nm/280 nm ratio with expected values between 1.8 and 2. Next, RNA samples were reverse-transcribed to cDNA, and primed single-stranded RNA using Random hexamer primer (ThermoFisher Scientific) reactions were performed according to the manufacturer's protocol. Two different primers were used with FAM fluorescent marker: murine Piezo-1 (P17113-005D08, ThermoFisher Scientific) and Piezo 2 (P17113-005D09, ThermoFisher Scientific) primers for BV2, murine microglia and primary mouse trigeminal culture cells, and human Piezo-1 (P17113-005D10, ThermoFisher Scientific) and Piezo 2 (P17113-005D11, ThermoFisher Scientific) for human post mortem cells, iPSC-derived human microglia and sv40 cell line (human immortalized microglia). PCR amplification of the cDNA was quantified using StepOnePlus Real-Time PCR System (ThermoFisher Scientific). Each sample had 3 replicates.

Calcium Imaging

To study the calcium influx evoked by native Piezo1 Rs expressed in microglia cells the present inventors used short Yoda1 applications (2 s, 50 μM if not otherwise specified). For this purpose, human IPSO derived microglia cells and SV-40 (immortalized human microglia) cells were seeded on 7 mm cover slips 1 day prior to the imaging experiment. Cells were loaded with the calcium-sensitive fluorescent dye Fluo-4AM (5 μM) for 30 min at 37° C. Followed by wash-incubation for 15 min in Ab 20 μM, 50 μM GdCI and basic solution (BS) as a control at 37° C. The basic solution containing the following (BS, in mM): 2.5 KCl, 152 NaCl, 10 glucose, 2 CaCl2, and 10 HEPES at pH 7.4. Fluorescence was visualized using monochromatic light source (TILL Photonics GmbH) using Ex/Im 494/5006 and a CCD camera (SensiCam). Chemicals were applied using a fast perfusion system (RSC-200, BioLogic Science). To quantify the difference in the amplitude of calcium transients, the ratio values were normalized by subtracting a baseline and further divided by the amplitude of the calcium transient to the 2 sec application of the ionomycin. Data were pre-analyzed offline using the FEI offline analysis (TILL Photonics), and further analysis was automatized using MatLab. Quantitative data were expressed as means±SEM unless otherwise stated. Number of experiments is indicated by n. Significance assessed with the ANOVA test or Mann-Whitney t test for non-parametric data. Statistically significant differences were set at*p<0.05 and **p<0.01.

Cell Culture Experiments

SV-40 immortalized human microglia cell lines. For imaging experiments, SV-40 immortalized human microglia cell line which endogenously express Piezo-1 and Piezo2Rs was used. SV-40 cell line was constantly maintained. Cells were grown in DMEM+GlutaMAX (Dulbecco's Modified Eagle Medium, Gibco) supplemented with 10% FBS (Gibco) and 1% Str/Pen in culture T-25 flasks (Starstedt) preliminary coated for 1 hour with collagen 1 (rat tail, Gibco).

Trigeminal neuronal culture. Trigeminal neurons culture was prepared as previously described (Abushik P A et al. 2017). In Brief, trigeminal ganglia were isolated from P10 Wistar rats and enzymatically dissociated at 37° C. for 15 min under continuous mixing (850 rpm) in trypsin (0.25 mg/mL, Sigma-Aldrich Co) with collagenase type I (760 U/mL, Sigma-Aldrich Co) solution. Next, cells were plated on poly-L-lysine pre-coated coverslips (0.2 mg/ mL, Sigma-Aldrich Co) and cultured in F12 Nutmix+GlutaMAX medium (Gibco Invitrogen) supplemented with FBS 10% (Gibco Invitrogen) at 37° C. with 5% CO2 for 48 h prior measurements.

Apoptosis assay in N2a cells. N2a cells were treated with 5 μM Yoda1 or vehicle for three consecutive days and exposed to 1% hypoxia as described above for 24 h before collecting the cells for apoptosis assay. The apoptotic cells were labeled with APC Annexin Ready Flow -dye (1:12.5 dilution, Invitrogen, Carlsbad, Calif., USA) in Annexin V Binding Buffer (10 mM HEPES, 150 mM NaCl, 2.5 mM CaCl2 in PBS, pH 7.4). 4′,6-Diamidino-2-Phenylindole, Dilactate (DAPI; Invitrogen, Carlsbad, Calif., USA) staining was added at 1:3300 dilution to label late apoptotic and necrotic cells. The samples were run with CytoFLEX S instrument (Beckman Coulter Life Sciences, Indianapolis, Ind., USA), and the results were analysed with CytExpert software (version 2.3.0.84, Beckman Coulter Life Sciences, Indianapolis, Ind., USA). The results of hypoxic samples were normalized to corresponding normoxic samples.

Measurement of neurite outgrowth in primary cortical neurons. Primary cortical neuron cultures were prepared from mouse embryos of embryonic day 15. The cortices were dissected, and tissue dissociated with 0.0125% trypsin (for 15 min at 37° C., Sigma-Aldrich, St. Louis, Mo., USA). After trypsin inactivation and washing the cells were counted and plated on 48-well-plates (coated with poly-D-lysine, Sigma-Aldrich, St. Louis, Mo., USA) at a density of 125 000 cells/well or on 6-well-plates at a density of 1.8 million cells/well in Neurobasal media supplemented with 2% B27 and 500 μM L-glutamine (all ThermoFisher Scientific, Waltham, Mass., USA) and 10 μg/ml of gentamicin (Sigma Aldrich, St. Louis, Mo., USA). On day 3 or 5 in vitro (DIV) the neurons were fed by changing 50% of media. The neurons were placed in IncuCyte® S3 Live Cell Analysis System (Essen

BioScience Ltd., Hertfordshire, UK) on DIV 0 and treated with 5 μM Yoda1 (Tocris, Bio-Techne Ltd., Abingdon, Oxfordshire, UK) once on DIV 0 or daily until DIV 7, and images were taken every 8 hours for the analysis of neurite length and branch points.

Differentiation of microglia. Differentiation of microglia was carried out as previously described with minor modifications (Abud et al., 2017). Shortly, on DO, iPSCs were dissociated to single cells with 0.5 mM EDTA or Accutase and were replated at a density of 6-,000-16,000 cells/cm2 on Matrigel in E8, 0.5% penicillin/streptomycin (P/S, 50 IU/50 mg/mL), 5 ng/ml BMP4, 25 ng/ml activin a (both from Peprotech or Miltenyi Biotec), 1 μM CHIR 99021 (Axon or Stem Cell Technologies) and 10 μM Y-27632 to induce mesodermal differentiation. To increase differentiation efficacy, the cells were maintained in low oxygen at 5% O_{2, 5}% CO₂, 37° C. On D1, the medium was replaced with a lower concentration of 1 μM Y-27632. After 48 hours on D2 the medium was changed to differentiation base medium (dif-base) containing DMEM/F-12, 0.5% P/S, 1% GlutaMAX™, 0.0543% sodium bicarbonate (all from Thermo Fisher Scientific), 64 mg/I L-ascorbic acid and 14 μg/lsodium selenite (both from Sigma). The dif-base was supplemented with 100 ng/ml FGF2, 50 ng/ml VEGF (both from Peprotech), 10 μM SB431542 (Selleckchem or Stem Cell Technologies), and 5 μg/ml insulin (Sigma) to induce the formation of hemogenic endothelium. On D4, the media was replaced by dif-base supplemented with 5 μg/ml insulin, 50 ng/ml FGF2, VEGF, IL-6 and TPO, and 10 ng/ml IL-3 and SCF to support the generation and proliferation of EMP. From now on, the cells were maintained in a normoxic incubator. Fresh EMP medium was changed daily until D8, when floating round EMPs were collected from the top of the monolayer. After centrifugation 300×g for 5 min, 350,000 cells/ml were transferred to ULA dishes (Corning) in microglial medium containing IMDM (Thermo Fisher Scientific), 0.5% P/S and 10% heat inactivated FBS (Biowest) or DMEM/F12, 0.5% N2, 0.5% B27 supplemented with 5 μg/ml insulin, 5 ng/ml MCSF and 100 ng/ml IL-34 (both from Peprotech). On D10, the cell suspension was changed by centrifuging and 350 000 cells/ml were seeded back to ULA dishes in microglial maturation medium supplemented with 10 ng/ml MCSF and 10 ng/ml IL-34. This medium was changed similarly every second day until D16, when the cells were detached from ULA-dishes with Accutase and replated on PDL-coated (Sigma) nunclon cell-culture treated plates (Thermo Fisher Scientific) in desired densities for experiments. Half of the maturation medium was changed daily until D23-D24 when experiments were performed.

Phagocytosis assay. To resolve if Piezo1 Rs activation can influence important function of microglia-phagocytosis the present inventors used hiPSC differentiated microglia cells for the live-cell analysis system (IncuCyte® S3, Sartorius) with bio-particles with pH sensitive-conjugated probes (Thermo Fisher Scientific). Cells were plated in 96 well plates and pre-treated 24h prior to the assay with testing compounds: vehicle (IMEM, Iscove's Modified Dulbecco's Medium, Gibco) with 0.5% P/S+IL-34 (Peprotech)+MCSF (Macrophage colony-stimulating factor, Peprotech), LPS 20 ng/mL (Escherichia Coli, serotype O111:64, Sigma), 5 μM Yoda1 (Tocris), 50 μM Gadolinium chloride (Tocris), LPS+Yoda1 (20ng/mL, 5 μM), LPS+Gadolinium (20ng/mL, 50 μM) and LPS+Yoda1+Gadolinium (20 ng/mL, 5 μM, 50 μM). Together with the pre-treatment pHRodoTM green zymosan bioparticles conjugate were added for phagocytosis (Thermo Fisher Scientific). Followed by the imaging in live-cell analysis system every 15 minutes for 5 hours. Further, fluorescence levels are measured and compared using Incucyte S3 software (Essen Bioscience).

Cytokine measurement. The impact of Piezo1Rs effect on the capacity of the iPSC derived microglia to release cytokines was evaluated using cytokine bead assay (CBA, PD Pharmingen)). Shortly, prior to the experiment IPSCs were plated in 24-well plate at 70,000 cells/cm² and cultured for 48 hours. Cells were cultivated in differentiation medium and treated with 20 ng/ml LPS or 5 μM Yoda1, 50 μM Gadolinium chloride, LPS+Yoda1 (20 ng/mL, 5 μM), LPS +Gadolinium (20 ng/mL, 50 μM) and LPS+Yoda1+Gadolinium (20 ng/mL, 5 μM, 50 μM). Next, media were collected, centrifuged to remove any cell debris, and the supernatant was frozen at −70° C. Cytokines were analyzed with cytometric bead array mouse inflammation kit (BD Biosciences) according to manufacturer's instructions.

Cytotoxicity assay. The cytotoxicity of in vitro cultures was assessed with Cytotox Green assay (Essen Bioscience) using Incucyte S3 Live-Cell Analysis System (Essen Bioscience, Ann Arbor, Mich., USA, #4647). Microglia were seeded for the assay at density of 15 000 cells per well on poly-D-lysine (PDL, Sigma, #P0899) coated Nunclon (Thermo Scientific, #167008) or ImageLock 96-well plates (Essen Bioscience, #4379). On the assay day, cells were treated with 0.1-20 μM Yoda1 (Tocris, #5586) in PM medium supplemented with 250 nM Cytotox Green reagent (Essen Bioscience, #4633). Equal volume of DMSO (Sigma, #D2650) was used as a vehicle and 200 μM MPP+(Sigma, #D048) was applied as a positive control. Cells were live-imaged in IncuCyte S3 at 37° C. and 5% CO2 every 3 hours for 3 days to obtain cell death over time. One to two 10× images per well were captured using the phase-contrast mode to obtain confluency and the green fluorescence mode to obtain fluorescent intensity. Quantification was performed using IncuCyte S3 Software (2019B). Green fluorescence was separated from background with a top-hat thresholding and all settings were kept constant between different conditions. As an outcome measure, green integrated intensity (GCU) per image was divided by confluence area per image at each time point and the data were normalized to maximum values of positive control or vehicle to combine mean group values from multiple experiments in the same figures.

Animal Experiments

All animal work was approved by the Animal Care and Use Committee of the University of Eastern Finland (Kuopio) and performed according to the guidelines of National Institutes of Health for animal care.

To evaluate the effect of microglial Piezo1 Rs mechanosensor in vivo the present inventors used 5-month-old transgenic SXFAD male mice (Jackson Laboratories, Bar Harbor, Me., US) which were randomly divided into 3 groups. 5xFAD mice express human APP and PSEN1 transgenes with a total of five AD-linked mutations: the Swedish (K670N/M671L), Florida (1716V), and London (V7171) mutations in APP, and the M146L and L286V mutations in Presenilin-1 (PSEN1). These widely used mice recapitulate many AD-related phenotypes and have a relatively early and aggressive presentation. Amyloid plaques, accompanied by gliosis, are seen in mice as young as two months of age. Amyloid pathology is more severe in females than in males. Neuron loss occurs in multiple brain regions, beginning at about 5 months in the areas with the most pronounced amyloidosis. Mice display a range of cognitive and motor deficits. The animals were implanted with the intraventricular cannulas. Briefly, surgical anesthesia was induced using 5% isoflurane and maintained with 1.8% isoflurane (in 30% 02/70% N20). The temperature of the animals was maintained at 37±0.5° C. using a thermostatically controlled heating blanket with a rectal probe (Pan Lab, Harvard Apparatus). After the skull was exposed a small hole approximately 1 mm in diameter was drilled into the left hemisphere of the skull using following coordinates: m/l (medial/lateral) +1.1 mm, a/p (anterior/posterior) −0.3 mm, d/v (dorsal/ventral) −2.0 mm. Then the specially designed chronic cannula (Cannula infusion system; Plastic1, Preclinical research components) were mounted true the drilled whole using and fixed on the mouse head using dental mount. After implantation animals were placed in individual cages to recover for 48h, following by cannula infusions of Yoda1 (0.05 μg/μl), GdCl (0.15 μg/μl) or Saline+1% DMSO once a day for next 2 weeks with 2 day break every 5 days/injections (total 10 day-injections). The mice were infused with 5 μl previously stetted drugs. Next, mice were euthanized 6h after last infusion for tissue collection. The mice were anesthetized with and overdose of Avertin followed by transcardial perfusion with heparinized saline (2500 IU/L). The ICV infused brain hemisphere were removed and post-fixed in 4% PFA followed by cryoprotection in 30% sucrose.

Brain sample preparation and Immunohistochemistry. The left hemisphere (injected) of the brain were frozen in liquid nitrogen and cryosectioned into 20-μm sagittal sections and stored in the antifreeze solution. Six consecutive sagittal brain sections at 400 μm intervals were selected for immunohistological staining from each mouse. Next, Aβ deposits and microglial were detected. The present inventors used primary antibody specific to human Aβ4-10 amino acids (WO2, Sigma Millipore, 1:1000 dilution) and lba-1 protein specific antibody (Iba1, Wako, 1:250 dilution) overnight at RT and further visualized by fluorescent Alexa 568 and Alexa 488 secondary antibodies (1:500 dilution, ThermoFisher Scientific) correspondingly. For quantification of Aβ and Iba1 immunoreactivities, the stained sections were imaged using 10× magnification in Zeiss Axio ImagerM.2 microscope equipped with Axiocam 506 mono CCD camera (Carl Zeiss, Oberkochen) running ZEN software (Carl Zeiss) for tailing and stitching of the images. Cortical and hippocampal Aβ and Iba1 immunoreactivities were quantified from 6 stained sections at 400 μm intervals per animal using MatLab code (MathWorks, MatLab 2017b). The accuracy of the analysis was confirmed by re-analyzing part of images using ImageJ 1.50i software. The data is represented as mean±SEM.

Permanent middle cerebral artery occlusion for modelling ischemic stroke and treatment with Yoda. Balb-c mice were subjected to permanent middle cerebral artery occlusion (pMCAo). The anaesthesia was induced by 5% isoflurane in 30% oxygen and 70% nitrogen as a carrier gas and maintained with 2% isoflurane during the surgical operation. The temperature was kept constant (37 ±1° C.) using the heating blanket connected to the rectal probe (Harvard apparatus, PanLab, Barcelona, Spain). The temporal muscle was detached from the scull and a hole (1 mm of diameter) was drilled to temporal bone. After removing the dura the exposed left MCA was lifted and occluded using a thermocoagulator (Aaron Medical Industries Inc., Clearwater, Fla., USA). The success of the occlusion was confirmed by cutting the artery, after which the temporal muscle was placed back on top of the hole and the skin was sutured. The mice were returned to their home cages to recover from the surgery. The sham operated animals were handled similarly, except for the occlusion of the MCA. Yoda1 (Tocris, Bio-Techne Ltd., Abingdon, Oxfordshire, UK; 1.78 mg/kg) was delivered intravenously (i.v.), dosing frequency being similar to that of HX600 in study I. Yoda1 stock of 50 mM was first prepared in dimethyl sulfoxide (DMSO; Sigma-Aldrich, St. Louis, Mo., USA), which was then diluted with 0.9% sterile saline (Baxter, Deerfield, Ill., USA) to obtain solution of desired concentration. Vehicle solution was prepared by diluting corresponding amount of DMSO into saline. Also, other solvents than DMSO can be used.

Adhesive removal test for evaluation of sensory motor functions. pMCAo induced sensorimotor deficits were tested using Adhesive removal test as previously described (Loppi et al. 2017). For Adhesive removal test each mouse was given four training sessions before induction of ischemia. In the first training the mice were habituated to the testing environment without adhesive tapes, and in second and third trainings the mice were placed into the test box with adhesives. The fourth training session took place one day before induction of ischemia and was used to record the baseline time for sensing and removing the batches. The actual tests were carried out at 1 dpi and 3 dpi before the MRI.

Lesion size measurement. The lesion volume was measured in vivo at 24 h and 72 h post ischemia by magnetic resonance imaging (MRI) using a vertical 9.4 T Oxford NMR 400 magnet (Oxford Instrument PLC, Abington, UK). The mice were anesthetized with 5% isoflurane and the anesthesia was maintained with 1% isoflurane during the imaging procedure. Multislice T2-weighted images (repetition time 3000 ms, echo time 40 ms, matrix size 128×256, field of view 19.2×19.2 mm², slice thickness 0.8 mm and number of slices 12) were taken and the obtained images were analyzed with in-house made Aedes software under the Matlab environment (Math-works, Natick, Mass., USA).

Red Blood Cell (RBC) Ca2+ Flux

The blood samples (50-100 μl) were collected from 5xFAD mice at different ages (13-15 weeks, 15-17 weeks, 17-19 weeks, 19-21 weeks and 21-23 weeks of age) for analysis of RBC Ca2 flux. Shortly, the blood samples were collected in Eppendorf tubes with sodium citrate to prevent coagulation. Then the samples were filtered through 100 μm filters to remove possible contaminants and aggregates, followed by two times wash with PBS (5 mins, 300g, RT) to remove plasma. 50 μl of red blood cells were transferred to dean tube and resuspended in 200 μl PBS (250 μl total volume of cell suspension). 250 μl of Fluo4 stock solution was added and cells were incubated for 30 minutes at 37° C. in dark. Cell suspension was then washed twice with PBS following with one wash with HBSS (5 mins, 300 g, RT). Supernatant was carefully removed with pipette and 50 μl pellet was resuspended in 450 μl of HBSS to form 10% cell suspension. 10μl of 10% cell suspension was transferred to 410 μl of RPMI/FBS (10:1). The Ca2+ flux was recorded by CytoFlex at high 60 μl/min flow rate. Recording was started at 20 sec run after the flow rate is stabilized, After 20 sec recording (baseline fluorescence), the Yoda1 at final concentration 10 μM was applied, at 4 min 20 sec time point (4 minutes after Yoda1 is added) ionomycin at 10 μM final concentration is added as positive control and sample is continued running until 6 min were up. Mean fluorescence intensity was measured using gates at different time points—baseline (before application of Yoda1 10 μM), at 1 min, 2 min, 3 min, at 4 min of recording.

### Example 1

### Piezo1 and Piezo2 are Mechanosensory Channels in Microglia Cell Types

In the present invention, Piezo receptor was discovered in human induced pluripotent stem cell (hIPSC)-derived microglia. This clinically highly relevant model recapitulates microglial phenotype at the level of development, function and transcriptome as well as expression of microglial markers giving us an unprecedented opportunity to study microglia in human context.

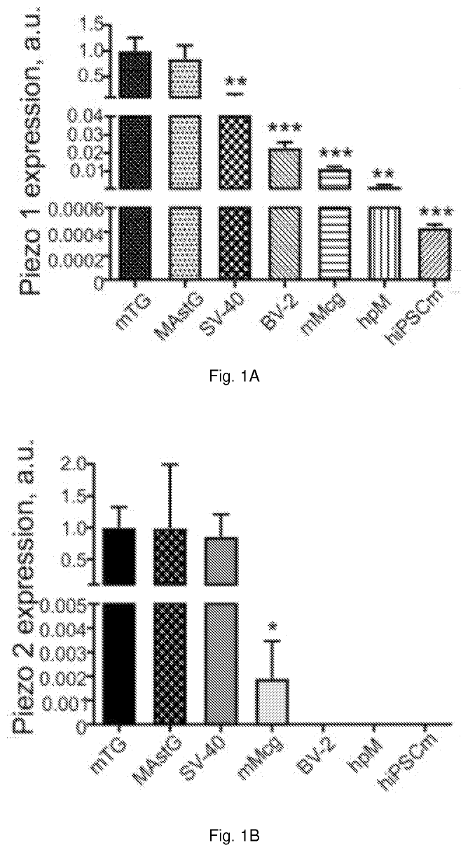

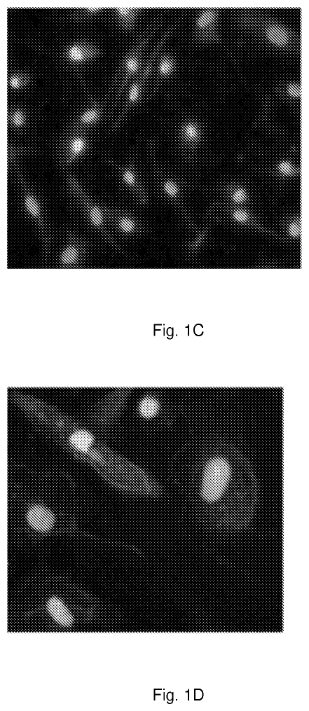

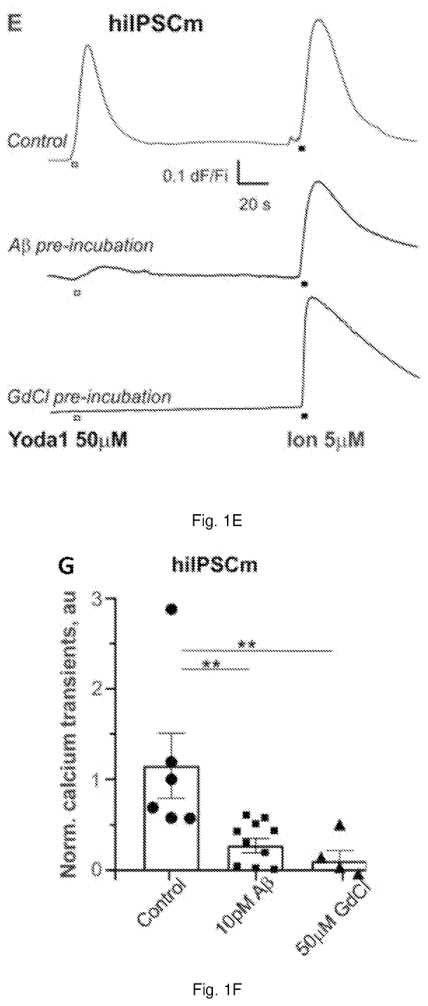

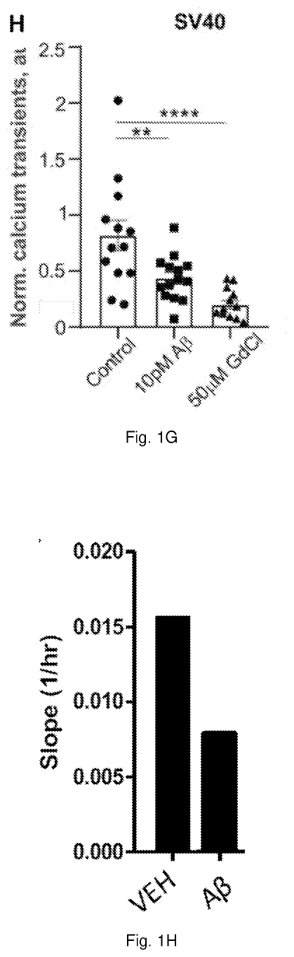

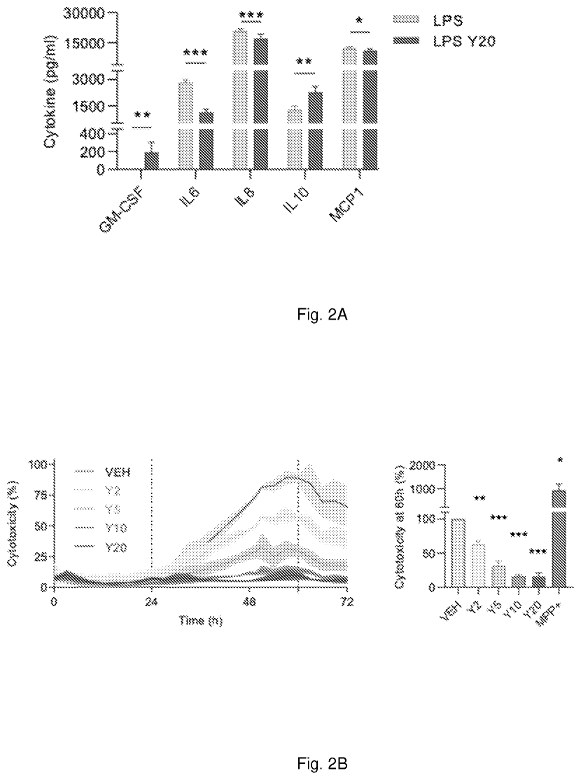

The qRT-PCR data revealed expression of the mechanosensitive Piezo1Rs in all types of the microglia tested. To sufficiently quantify presence of the Piezo receptors expression level the present inventors normalized their data to Piezo receptors expression in mouse trigeminal neurons (mTG, FIGS. 1A, 1B), which are known to contain both types of receptors. The present inventors found that mouse astrocytes demonstrate high level of both Piezo mechanosensitive channels' expression (Piezo1: 0.8474±0.2551; Piezo2: 0.8576±0.2042, n=4, FIGS. 1A, 1B). Between tested microglial types only cell line SV-40 (Piezo1: 0,1188±0,0458; Piezo2: 0,9901±0,5015; n=4) and mouse microglia (Piezo1: 0.0114±0.0012; Piezo2: 0.0019±0.0008; n=4) demonstrated presence of both mechanosensitive receptors. However, the present data demonstrated that human postmortem microglia, human iPSC derived microglia and BV2 (immortalized murine microglia) cell line solely express Piezo1Rs (hpM: 0.0018±0.0008, n=4; hiPSCm: 0.000434±0.000003, n=4; BV2: 0,0229±0,0031, n=4). Consistent with the qPCR data the present inventors demonstrate Piezo1Rs morphological expression by IHC in hilPSC derived microglia represented in FIGS. 1C and 1D.

### Example 2

### Yoda-1 Activated Piezo1 Ca²⁺ Influx in Human Microglia Cells and Reversed AR Effects

Following the first discovery of mechanosensory in microglia, the functional effects were tested in microglia of the Piezo1 Rs using Ca²⁺-imaging in hiPSC cells and SV-40 cell line. hiPSC derived microglia were chosen as closest by the expression level of Piezo1Rs cell line to human postmortem microglial levels and SV-40 cell line as one with the highest expression level. Apart from mechanical stimuli, Piezo1 Rs (but not Piezo2) channels could be co-activated by the small molecule called Yoda1. Using this unique opportunity to activate native Piezo1Rs mechanosensitive the present inventors used 50 μM Yoda1 diluted in basic solution applied with fast solenoid-valve based perfusion system (Biologic 2000) that additionally generates small hydraulic pressure in the start of the application. It was demonstrated that despite low expression of Piezo1 Rs in microglia compare to TG neurons and astrocytes, these cells express enough native Piezo1Rs on the membrane to activate strong Ca-influx in both tested microglial cell lines (FIG. 1E top trace).

It was further assessed if the soluble Aβ low concentrations as in early AD model can affect native Piezol-evocked Ca²⁺influx in human microglia. Based on preliminary tests even low Aβ concentrations activated Ca²⁺influx, thus short (15min) pre-incubation protocol was used to avoid it's overlapping with Yoda1 activated Piezo1 Rs stimuli. Control hiPSC microglia was pre-incubated for same time in BS. Control group of cells demonstrated strong Ca²⁺ transients evoked by Yoda1 short (2 s) application (1.15±0.36, n=6; FIG. 1E).

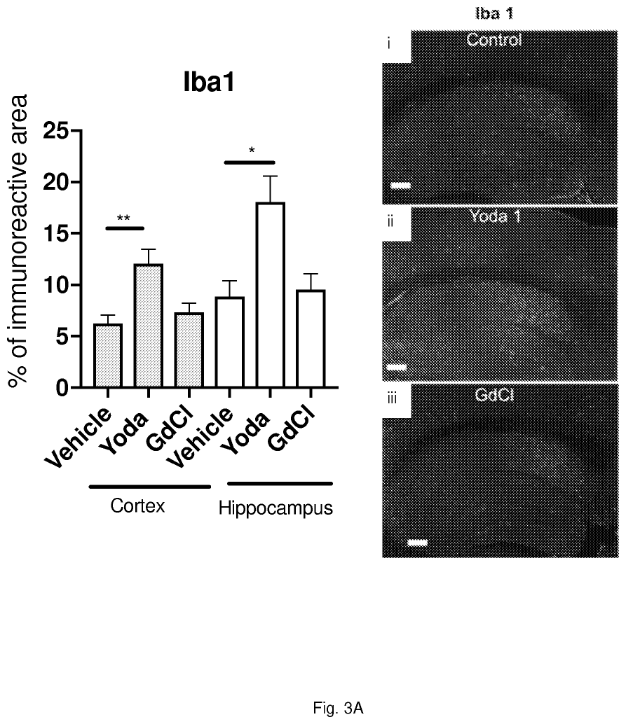

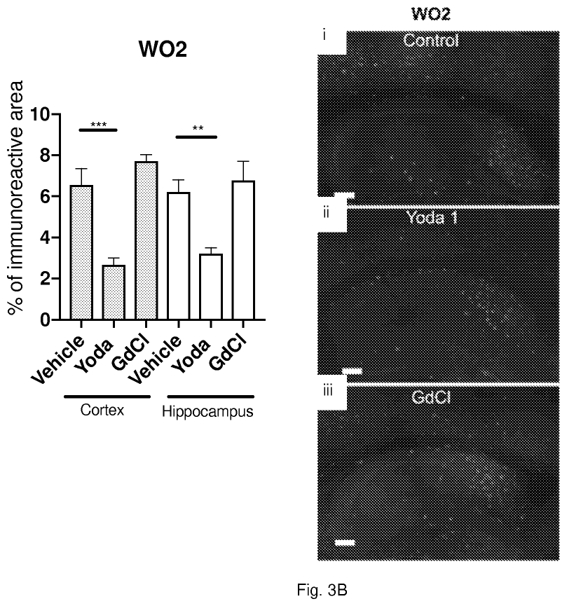

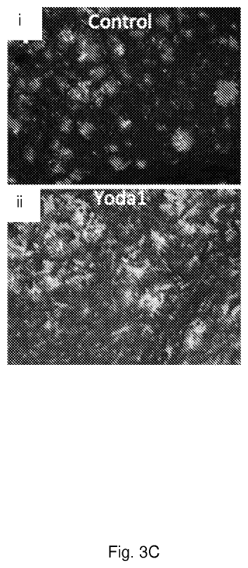

Pre-incubation of microglia-like cells with 20 μM soluble Aβ (diluted in BS) strongly inhibited Yoda1-activated Piezo1Rs Ca²⁺influx (0.27±0.08, p=0.0073, n=11; FIGS. 3E, 3F). Next, pre-incubation in non-specific Piezo1 inhibitor GdCI (50 μM in BS), which inhibited Ca²+transients by 90% (0.10±0.1, p=0.0073, n=5; FIGS. 1E, 1G), was used.

A similar protocol of the pre-incubation in Ca²⁺ imaging experiment on SV-40 sell line, which the present inventors found to express highest from other microglia Piezo1Rs level and even higher level of Piezo2Rs, was next used. The data demonstrated 50% inhibition of Ca²⁺transients by Aβ pre-incubation of SV-40 cell line (0.82±0.14, n=13 vs 0.44±0.05, n=14; p=0.0085; FIG. 1H), which can be explained by presence of possibly of Aβ not inhibited part of the Piezo2 mechanically evoked (by hydraulic perjure by the fast perfusion system) Ca²⁺ input as SV-40 cell line expresses both types of Piezo channels. This hypothesis is supported by the result of strong inhibition of the Yoda1 activated Ca²+transients in SV-40 cells by pre-incubation of GdCI (0.82±0.14, n=13 vs 0.17±0.04, n=13; p=0.00009). Further to test the hypothesis, the present inventors tested range of Ca²+ transients evoked by BS applications alone using their fast perfusion system on control SV-40.

This data suggests that low concentrations of soluble Aβ strongly inhibit native Piezo1Rs in human microglia, what can lead to their sensitivity to mechanical stimuli in the brain via inhibited Ca²⁺ signaling.

### Example 3

### Activation of Yoda1 Renders Microglia Less Pro-Inflammatory

Piezo activation and inhibition had functional consequence in microglia, as specific activation of Piezo by Yoda1 significantly calmed down microglial pro-inflammatory activation (increased pro-inflammatory cytokine secretion) induced by LPS suggesting that activation of Piezo beneficially modulates microglial functions (FIG. 2A). Moreover, activation of Piezo by Yoda1 significantly protected the iPSC-microglia from cytotoxicity (FIG. 2B)

### Example 4

### Yoda1 is Enhancing Microglial Phagocytosis In Vivo

To provide proof-of-concept of the impact of Yoda1 on enhancing microglial phagocytosis, 5xFAD transgenic mice modeling Alzheimer's disease were treated with Yoda1 or piezo inhibitor Gadolinium (GdCI) for 2 weeks. The brains were analyzed by immunohistochemical stainings for the brain Aβ burden and microgliosis. The results demonstrate that Yoda1 treated mice show significantly increased Iba1 immunoreactivity (FIG. 3A), significant reduction in the extent of brain Aβ load both in cortex and in the hippocampi (FIG. 3B) and enhanced microglial accumulation near the Aβ deposits (FIG. 3C)

### Example 5

### Primary Neurons Express Piezo During Their Development

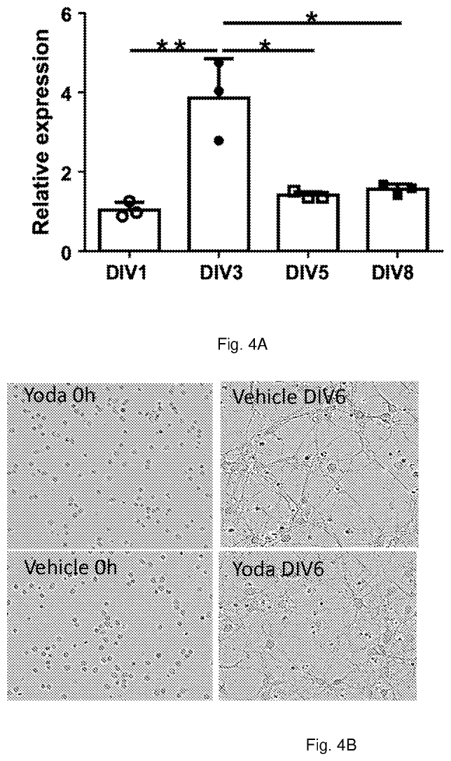

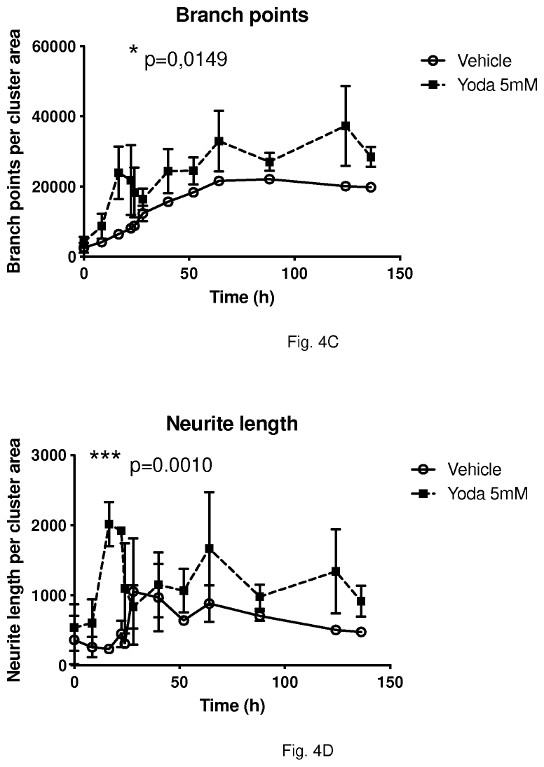

To provide evidence that piezo1 is expressed in brain microglia, immunohistochemical stainings for piezo were carried out. Surprisingly, piezo was also expressed in neurons, also in the hippocampal dentate gyrus where neurogenesis is known to take place. This let the present inventors to test what is the impact of Yoda1 induced piezol activation specifically in neurons. The data show that primary neurons express piezo during their development (FIG. 4A). Thus, when primary neurons were treated with Yoda1 the present inventors were able to show that Yoda1 treated neurons develop significantly more extensive branching (FIGS. 4B and 4C) and neurite length (FIG. 4D) compared to their vehicle treated control cells. This implicates that Yoda1 is able to enhance neuronal development.

### Example 6

### Yoda1 Exerts Protection Also in Conditions of Ischemic Stroke

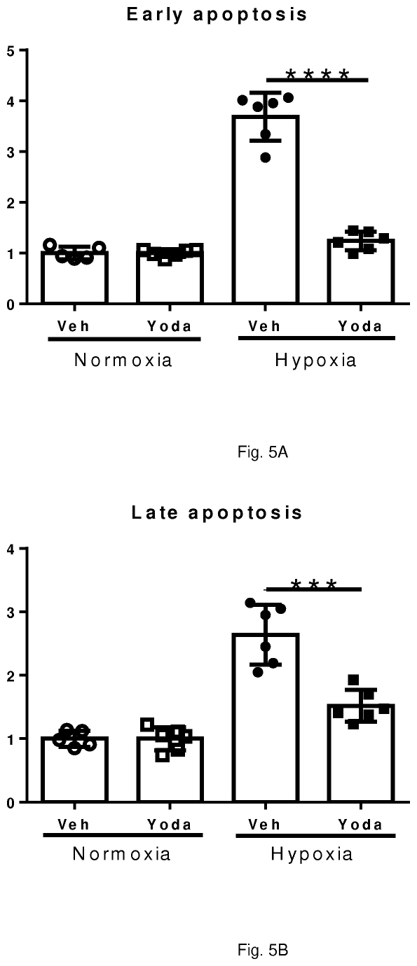

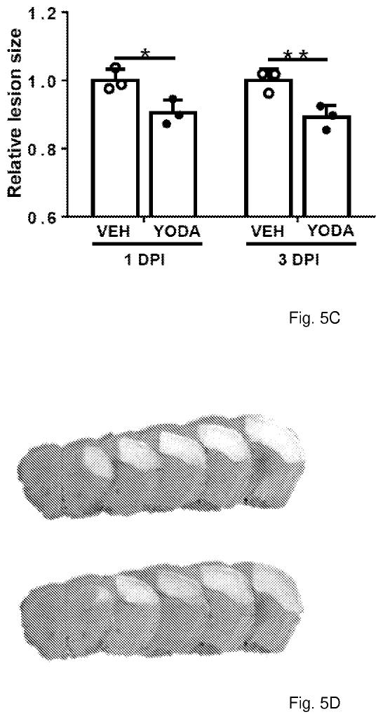

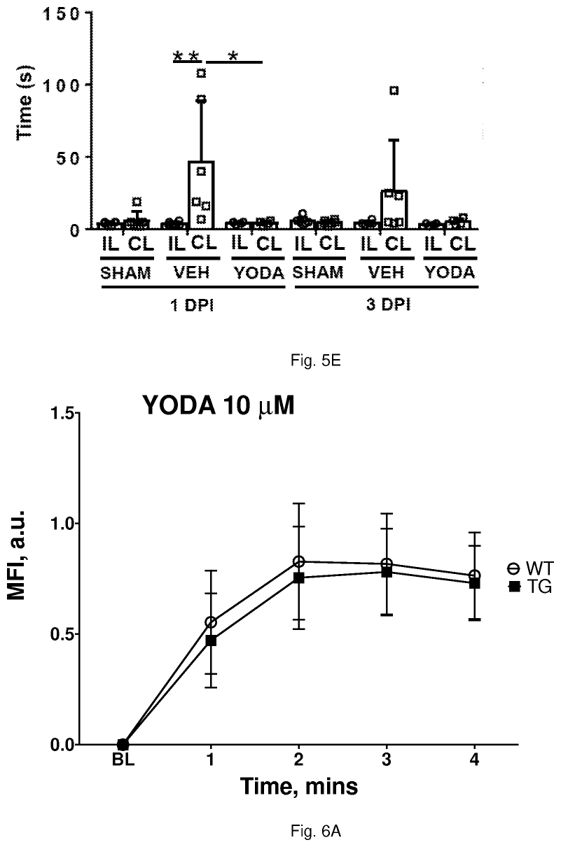

Since modulation of microglial functions and enhancement of neurogenesis have been suggested as potential therapeutic approaches, it was hypothesized that Yoda1 exerts protection also in conditions of ischemic stroke. The present inventors first analyzed whether Yoda1 prevents hypoxia-induced apoptosis and were able to demonstrate that Yoda1 is efficient in preventing both early and late apoptosis induced by hypoxic conditions in vitro (FIGS. 5A, 5B). To evaluate whether Yoda1 is protective against ischemia induced sensorimotor deficits, balbc-mice were subjected to permanent middle cerebral artery occlusion and analyzed the ability of Yoda1 treatment to prevent the ischemia induced deficits in adhesive removal test. Indeed, Yoda1 treated mice were comparable to sham operated controls in this sensory motor test. Yoda1 was administered immediately after stroke and thereafter once a day during three consecutive days. Lesion size was measured with MRI at 1 DPI and 3 DPI. The Yoda1 treated mice showed significantly smaller lesion size at both timepoints (FIG. 5C). Representative MRI images from vehicle (upper images) and Yoda1 treated mice (lower images), respectively (FIG. 5D). Yoda1 treated ischemic mice show no apparent deficits in their ability to sense adhesive patches post stroke (FIG. 5E). All data are presented as mean+/−SEM with *p<0.05, **p<0.01, ***p<0.001 as analyzed by t test or two-way ANOVA followed by Bonferroni post hoc test.

### Example 7

### Piezo Receptor Activation by Yoda1 Acts as a Functional Biomarker in Alzheimer's Disease

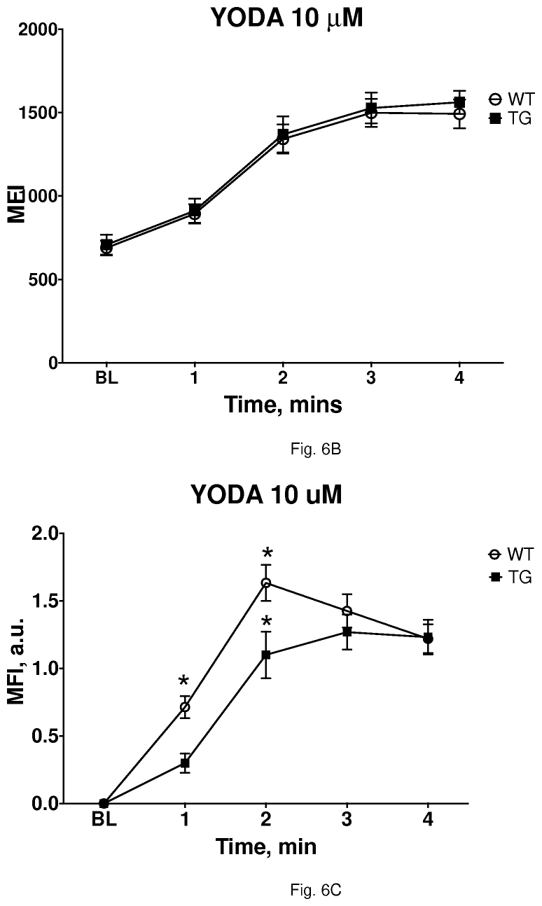

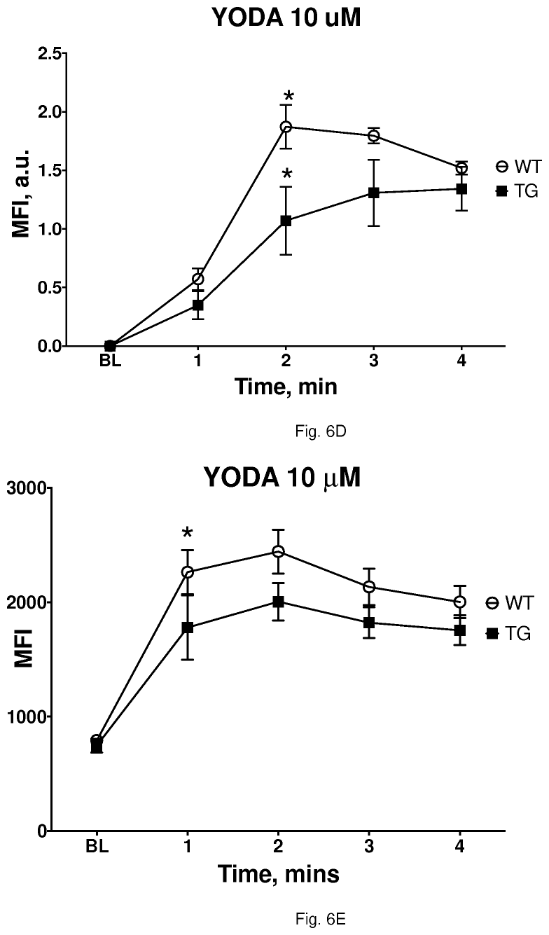

Since piezo receptor can be blocked aβ-peptides, the present inventors hypothesized that its functionality is reduced in conditions of AD. Indeed, the present inventors demonstrate that Yoda1 elicit responses in blood are significantly lower in transgenic (TG) 5xFAD mice compared to their age-matched wildtype (WT) controls (FIGS. 6A-6E). Blood samples were drawn from AD transgenic mice (TG) of different age and and their wildtype controls (WT) for analysis of red blood cell calcium flux induced by yoda1. The cells were stimulated with 10 uM of piezo agonist Yoda1 and run on cytoflex for their Calcium responses. The blood cells from TG mice responded at lower intensity compared to age-matched WT controls at 17-23 weeks of age suggesting that Yoda-response may act as a functional biomarker in AD.

The main result is that already at this early stage of AD pathology the murine Piezo channels reduced their activity as follows from the reduced calcium flux in RBCs activated by the Piezo1 agonist Yoda1. This method includes several steps: 1) The small volume (100 μI) of blood sample with citrate to prevent coagulation is washed with PBS and after centrifugation the RBC pellet is resuspended in PBS; 2) This sample is loaded with the calcium indicator Fluo4AM; 3) After loading, cells are washed, the supernatant is removed and the pellet is resuspended in HBSS to get 10% cell suspension; 4) The sample is transferred to RPMI/FBS; 5) The sample is used for recording of intracellular calcium dynamics in the time-lapse mode by using CytoFlex machine. 6) Mean fluorescence intensity is measured at the baseline and at different time points after application of Yoda1. This data suggests that Piezo receptor activation is dysfunctional in AD and can be used as a functional biomarker for AD.

### Example 8

### Ca²⁺ Flux Method

The blood samples (50-100 μI) were collected in Eppendorf tubes with sodium citrate to prevent coagulation. Then the samples were filtered through 100 μm filters to remove possible contaminants and aggregates, following by two times wash with PBS (5 mins, 300 g, RT) to remove plasma.

After centrifuging the sample solution, red blood cells formed the pellet. 50 μl of red blood cells were transferred to clean tube and resuspended in 200 μl PBS (250 μl total volume of cell suspension). 250 μl of Fluo4 stock solution was added and cells were incubated for 30 minutes at 37° C. in dark.

Cell suspension was washed twice with PBS following with one wash with HBSS (5 mins, 300 g, RT). Supernatant was carefully removed with pipette and 50 μl pellet was resuspended in 450 μl of HBSS to form 10% cell suspension. 10 μl of 10% cell suspension was transferred to 410 μl of RPMI/FBS (10:1). The final volume was 420 μI which was sufficient for max 7 minutes recording by CytoFlex at high 60 μl/min flow rate.

The samples were recorded for 6 minutes. Recording was started at 20 sec run after the flow rate was stabilized. After 20 sec recording (baseline fluorescence), the Yoda1 at final concentration 10 μM was applied, at 4 min 20 sec time point (4 minutes after Yoda1 was added) ionomycin at 10 μM final concentration was added as positive control and sample was continued running until 6 min were up. Mean fluorescence intensity was measured using gates at different time points—baseline (before application of Yoda1 10 μM), at 1 min, 2 min, 3 min, at 4 min of recording.

### REFERENCES

Abud, E. M., Ramirez, R. N., Martinez, E. S., Healy, L. M., Nguyen, C. H. H., Newman, S. A., Yeromin, A. V., Scarfone, V. M., Marsh, S. E., Fimbres, C., et al 2017, “iPSC-Derived Human Microglia-like Cells to Study Neurological Diseases”, Neuron, vol. 94, no. 2, pp. 278-293.e9

Abushik P, Bart G, Korhonen P, Leinonen H, Giniatullina R, Sibarov D A, Levonen A L, Maim T, Antonov S M and Giniatullin R. Pro-nociceptive migraine mediator CGRP provides neuroprotection of sensory, cortical and cerebellar neurons via multi-kinase signaling. Cephalgia 2017 December; 37(14):1373-1383

Blumenthal, Nils, Hermanson Ola, Heimrich Bernnd and Shastri V. Prasad. 2014.

“Stochastic nanoroughness modulates neuron-astrocyte interactions and function via mechanosensing cation channels”. PNAS 111(45) 16124-16129.

Bollmann, Lars et al. 2015. “Microglia Mechanics: Immune Activation Alters Traction Forces and Durotaxis.” Frontiers in cellular neuroscience 9: 363.

Bron, Romke, Rhiannon J. Wood, James A. Brock, and Jason J. Ivanusic. 2014. “Piezo2 Expression in Corneal Afferent Neurons.” Journal of Comparative Neurology 522(13): 2967-79.

Cai, Zhiyou, M. Delwar Hussain, and Liang-Jun Yan. 2014. “Microglia, Neuroinflammation, and Beta-Amyloid Protein in Alzheimer's Disease.” International Journal of Neuroscience 124(5): 307-21.

Coste, B. et al. 2010. “Piezo1 and Piezo2 Are Essential Components of Distinct Mechanically Activated Cation Channels.” Science 330(6000): 55-60.

Coste, Bertrand et al. 2012. “Piezo Proteins Are Pore-Forming Subunits of Mechanically Activated Channels.” Nature 483(7388): 176-81.

dela Paz, Nathaniel G., and John A. Frangos. 2018. “Yoda1-Induced Phosphorylation of Akt and ERK1/2 Does Not Require Piezo1 Activation.” Biochemical and Biophysical Research Communications 497(1): 220-25.

Eijkelkamp, N et al. 2013. “A Role for Piezo2 in EPAC1-Dependent Mechanical Allodynia.” Nature Communications 4: 1682.

ElSheikh, Mona et al. 2017. “MR Elastography Demonstrates Unique Regional Brain Stiffness Patterns in Dementias.” American Journal of Roentgenology 209(2): 403-8.

Hammond, Timothy R., Daisy Robinton, and Beth Stevens. 2018. “Microglia and the Brain: Complementary Partners in Development and Disease.” Annual Review of Cell and Developmental Biology 34(1): 523-44.

Kim, Sung Eun et al. 2012. “The Role of Drosophila Piezo in Mechanical Nociception.” Nature 483(7388): 209-12.

Li, Jing et al. 2014. “Piezo1 Integration of Vascular Architecture with Physiological Force.” Nature 515(7526): 279-82.

Loppi S, Kolosowska N, Kärkkäinen 0, Korhonen P, Huuskonen M, Grubman A, Dhungana H, Wojciechowski S, Pomeshchik Y, Giordano M, Kagechika H, White A, Auriola S, Koistinaho J, Landreth G, Hanhineva K, Kanninen K, Maim T. HX600, a synthetic agonist for RXR-Nurr1 heterodimer complex, prevents ischemia-induced neuronal damage. Brain Behav Immun 2018 Oct; 73:670-681.

MaIm, Tarja M, Taylor R Jay, and Gary E Landreth. 2015. “The Evolving Biology of Microglia in Alzheimer's Disease.” Neurotherapeutics: the journal of the American Society for Experimental Neuro Therapeutics 12(1): 81-93.

Maneshi, Mohammad M. et al. 2018. “Enantiomeric Aβ Peptides Inhibit the Fluid Shear Stress Response of PIEZ01.” Scientific Reports 8(1): 14267.

Moeendarbary, Emad et al. 2017. “The Soft Mechanical Signature of Glial Scars in the Central Nervous System.” Nature communications 8: 14787.

Moshayedi, Pouria et al. 2014. “The Relationship between Glial Cell Mechanosensitivity and Foreign Body Reactions in the Central Nervous System.” Biomaterials 35(13): 3919-25.

Murphy, Matthew C. et al. 2016. “Regional Brain Stiffness Changes across the Alzheimer's Disease Spectrum.” Neurolmage: Clinical 10: 283-90.

Parkhurst, Christopher N. et al. 2013. “Microglia Promote Learning-Dependent Synapse Formation through Brain-Derived Neurotrophic Factor.” Cell 155(7): 1596-1609.

Perlmutter, L. S., S. A. Scott, E. Barron, and H. C. Chui. 1992. “MHC Class II-Positive Microglia in Human Brain: Association with Alzheimer Lesions.” Journal of Neuroscience Research 33(4): 549-58.

Smith, Jeffrey F et al. 2006. “Characterization of the Nanoscale Properties of Individual Amyloid Fibrils.” Proceedings of the National Academy of Sciences of the United States of America 103(43): 15806-11.

Velasco-Estevez, Maria et al. 2018. “Infection Augments Expression of Mechanosensing Piezo1 Channels in Amyloid Plaque-Reactive Astrocytes.” Frontiers in aging neuroscience 10: 332.

Wang, ShengPeng et al. 2016. “Endothelial Cation Channel PIEZO1 Controls Blood Pressure by Mediating Flow-Induced ATP Release.” The Journal of Clinical Investigation 126(12): 4527-36.

Wu, Jason, Amanda H Lewis, and Jörg Grandl. 2017. “Touch, Tension, and Transduction—The Function and Regulation of Piezo Ion Channels.” Trends in Biochemical Sciences, 42(1): 57-71.

1. A method of treating a neurodegenerative disease and/or neuroinflammatory disease, or a condition or disorder associated with a neurodegenerative disease and/or neuroinflammatory disease in a subject, the method comprising administering a Piezo agonist to the subject.

2. The method of claim 1, wherein the Piezo is Piezo1 or Piezo2.

3. The method of claim 1, wherein the Piezo is from a mouse or a human being.

4. The method of claim 1, wherein the Piezo agonist is for modulation of microglia.

5. The method of claim 4, wherein the microglia function to be modulated is motility, phagocytosis and/or cytokine release.

6. The method of claim 1, wherein the agonist is used for activating Piezo.

7. The method of claim 1, wherein the agonist is selected from the group consisting of Yoda1, Jedi1, Jedi2, and functional analogs thereof.

8. The method of claim 1, wherein the agonist is Yoda 1.

9. The method of claim 1, wherein amyloid-β accumulation is inhibited.

10. The method of claim 1, wherein the load of amyloid-β plaques is reduced.

11. The method of claim 1, further comprising administering to the subject at least one molecule selected from the group consisting of arachidonoylethanolamide, 2-arachidonoylglycerol, palmitoylethanolamide, oleoylethanolamide, and linoleoyl ethanolamide.

12. The method of claim 1, wherein the neurodegenerative disease and/or neuroinflammatory disease, or a condition or disorder associated with the neurodegenerative disease and/or neuroinflammatory disease is selected from the group consisting of Alzheimer's disease, stroke, Parkinson's disease, head trauma(s), cerebral amyloid angiopathies, spongiform encephalopathies, cerebral amyloid disease, and scrapie.

13. The method of claim 1, wherein the treatment is preventive or therapeutic treatment of a neurodegenerative disease and/or neuroinflammatory disease, or a condition or disorder associated with a neurodegenerative disease and/or neuroinflammatory disease.

14. (canceled)

15. (canceled)

16. (canceled)

17. The method according to claim 1, wherein the Piezo agonist is uscd in administered combination with at least one molecule selected from the group consisting of arachidonoylethanolamide, 2-arachidonoylglycerol, palmitoylethanolamide, oleoylethanolamide, and linoleoyl ethanolamide.

18. (canceled)

19. A method for determining a risk associated with development or presence of a neurodegenerative disease and/or neuroinflammatory disease, or a condition or disorder associated with said neurodegenerative disease and/or neuroinflammatory disease in a human subject, comprising the steps of:
a. providing a test sample and a control sample;
b. measuring baseline fluorescence intensity of the test sample and the control sample;
c. adding Piezo agonist to said test sample;
d. measuring fluorescence intensity of the test sample and the control sample; and
e. determining the difference of the fluorescence intensity of the test sample and the control sample, wherein the reduced fluorescence intensity of the test sample in step e as compared to the fluorescence intensity of the control sample is indicative of reduced Piezo receptor activity in said test sample and of risk for development or presence of a neurodegenerative disease and/or neuroinflammatory disease, or a condition or disorder associated with said neurodegenerative disease and/or neuroinflammatory disease in said human subject.

20. The method according to claim 19, wherein the test sample and control sample consist of red blood cells, serum, plasma or whole blood or blood mononuclear cells.

21. The method according to claim 20, wherein the test sample and control sample consist of red blood cells or blood mononuclear cells.

22. The method of claim 19, wherein the neurodegenerative disease and/or neuroinflammatory disease, or a condition or disorder associated with said neurodegenerative disease and/or neuroinflammatory disease includes at least one of the following Alzheimer's disease, Parkinson's disease, stroke, head trauma(s), cerebral amyloid angiopathies, spongiform encephalopathies, cerebral amyloid disease, and scrapie.

23. A method of modulating microglia function in a microglial cell comprising contacting the cell with a Piezo agonist.
